(19) United States
(12) Patent Application Publication
(10) Pub. No.: US 2020/0155327 A1
(43) Pub. Date: May 21, 2020

(54) SPACE BRIDGING PLATE AND MODULAR COMPONENTS

(71) Applicant: CTL Medical Corporation, Addison, TX (US)

(72) Inventors: Jon SUH, Ambler, PA (US); Sean SUH, Milltown, NJ (US)

(21) Appl. No.: 16/749,296

(22) Filed: Jan. 22, 2020

Related U.S. Application Data
(63) Continuation-in-part of application No. 16/291,278, filed on Mar. 4, 2019.
Application No. 16/291,278 is a continuation of application No. 15/244,868, filed on Aug. 23, 2016, now Pat. No. 10,219,912.
(60) Provisional application No. 62/270,141, filed on Dec. 21, 2015.
(60) Provisional application No. 62/795,414, filed on Jan. 22, 2019.

(51) Int. Cl. A61F 2/44

(52) U.S. Cl. CPC A61F 2/4455; A61F 2002/3093; A61F 2/447

(57) ABSTRACT

Disclosed are devices for the fixation and support of vertebrae, particularly spinal implant devices supporting and/or securing removed vertebral bodies of the spine.

### CROSS-REFERENCE TO RELATED APPLICATIONS

This application is a continuation-in-part of U.S. patent application Ser. No. 16/291,278, filed Mar. 4, 2019 and entitled “MODULAR PLATE AND CAGE ELEMENTS AND RELATED METHODS,” which is a continuation of U.S. patent application Ser. No. 15/244,868, filed Aug. 23, 2016 and entitled “MODULAR PLATE AND CAGE ELEMENTS AND RELATED METHODS,” which claims priority to and benefit thereof from U.S. Provisional Patent Application No. 62/270,141, filed Dec. 21, 2015, titled “MODULAR PLATE AND CAGE ELEMENTS AND RELATED METHODS,” the disclosures of which are each hereby incorporated herein by reference in their entireties. This application also claims priority to and benefit thereof from U.S. Provisional Patent Application No. 62/795,414, filed Jan. 22, 2019, titled “CORPECTOMY PLATE,” the disclosure of which is hereby incorporated herein by reference in its entirety.

### FIELD OF THE INVENTION

The present subject matter relates generally to devices for the fixation and support of vertebrae. In particular, the present subject matter relates to an implant device for supporting and/or securing vertebral bodies of the spine.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

Another technique for treating spinal disorders is a corpectomy or vertebrectomy, which is a surgical procedure that involves removing all or part of the vertebral body, often as a way to decompress the spinal cord and nerves or as a treatment for spinal metastases. Corpectomy is often performed in association with some form of discectomy. When the vertebral body has been removed, the surgeon typically performs a vertebral fusion to fill the space in the spinal column (which was occupied by the removed vertebral bone), which may include use of a block of bone taken from the pelvis or one of the leg bones or with a manufactured component such as a cage. Desirably, the bone graft will hold the remaining vertebrae apart, while the vertebrae grow together and fuse.

Current treatments for spinal disorders and/or other issues present a variety of surgical challenges. As such, there is need for further improvement, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with an aspect of the present subject matter, an implant device for the spine is provided. The implant device is for location between two vertebrae, such as two adjacent vertebrae separated by a vertebrae that has been removed during a corpectomy or vertebrectomy procedure. The implant can comprise a first plate having a first end, a second end, a longitudinal axis, and upper surface, and a lower surface, the first plate having at least two fixation holes extending from the upper surface to the lower surface, the first plate further comprising first and second arms extending outward from the lower surface of the plate, the first and second arms each including vertebral endplate engaging portions.

In various embodiments, an implant device for the spine is provided. The implant device is for location between two vertebrae, such as two adjacent vertebrae separated by a vertebrae that has been removed during a corpectomy or vertebrectomy procedure. The implant can comprise a first plate and an interbody device, the first plate having a first end, a second end, a longitudinal axis, and upper surface, and a lower surface, the first plate having at least two fixation holes extending from the upper surface to the lower surface, the first plate further comprising first and second arms extending outward from the lower surface of the plate, the first and second arms each including vertebral endplate engaging portions, the interbody device sized and configured for positioning between the two adjacent vertebrae. In some embodiments, the interbody device may be sized and configured to be connected to the first plate, while in other embodiments at least a portion of the interbody device can be positioned between the first and second arms.

In various embodiments, the interbody device or graft material (i.e., a bone block or other device) may be connected to the first plate and/or other implant components prior to insertion into the patient's anatomy, while in other embodiments, one or more components of the implant assembly may be individually inserted into the patient and then the full implant may be assembled in situ within the patient's anatomy.

A method for manufacturing an implant device as indicated above.

A method for manufacturing an implant device as set for within any of the details described with the present application.

An implant device for the spine as set for within any of the details described with the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 illustrates a portion of a patient's spinal column;

FIG. 2 illustrates an interbody device positioned within the patient's spinal column;

FIG. 3 illustrates a perspective view of an example of a cage, constructed according to the principles of the disclosure;

FIG. 4 illustrates a perspective view of another example of a cage, constructed according to the principles of the disclosure;

FIGS. 5A-5F illustrate examples of cages, constructed according to the principles of the disclosure;

FIGS. 6A-6F illustrate further examples of cages, constructed according to the principles of the disclosure;

FIGS. 7A-7B illustrate different views of an example of a cage, constructed according the principles of the disclosure;

FIG. 8 illustrates and an example of an interbody system, constructed according to the principles of the disclosure;

FIGS. 9A-9C illustrate examples of a modular cage, constructed according to the principles of the disclosure;

FIG. 10 illustrates further examples of a modular cage, constructed according to the principles of the disclosure;

FIGS. 11A-11F show top views of examples of interbody devices, constructed according to the principles of the disclosure;

FIGS. 12A-12J illustrate examples of interbody devices, constructed according to the principles of the disclosure;

FIGS. 13A-13C illustrate different views of an example of an interbody device, constructed according to the principles of the disclosure;

FIG. 14 illustrates an example of the interbody device of FIGS. 13A-13C installed between a pair of bony structures;

FIGS. 15A-15E illustrate different views of an interbody system that includes the cage of FIG. 4 and the interbody device of FIGS. 13A-13C;

FIGS. 16A-16C illustrate an example of the interbody system of FIGS. 15A-15D installed between a pair of bony structures;

FIG. 17 A illustrates a further example of an interbody device, constructed according to the principles of the disclosure;

FIGS. 17B-17E illustrate various views of an example of an interbody system that includes the plate device of FIG. 17 A; and

FIGS. 18A-18C illustrate an example of the interbody system of FIGS. 17B-17E installed between a pair of bony structures.

FIG. 19A illustrates a perspective view of one exemplary embodiment of a corpectomy support plate;

FIGS. 19B through 19G depict various views of the corpectomy support plate of FIG. 19A;

FIG. 20 depicts one exemplary embodiment of a corpectomy plate positioned in a simplified representation of a vertebral column;

FIG. 21 depicts views of various sized corpectomy plate embodiments;

FIG. 22A depicts a prior art 6-hole corpectomy plate construct;

FIG. 22B depicts the construct of FIG. 22A in a simplified representation of a vertebral column;

FIG. 23A depicts another exemplary embodiment of a corpectomy plate; and

FIG. 23B depicts the corpectomy plate of FIG. 23A in a simplified representation of a vertebral column.

### DETAILED DESCRIPTION OF THE INVENTION

The present subject matter relates generally to devices for the fixation and support of vertebrae. In particular, the present subject matter relates to an implant device for supporting sections of the vertebral column where one or more vertebral bodies and/or portions thereof have been resected and/or removed.

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain, and can sustain permanent neurological damage if the conditions are not treated appropriately.

One technique of treating spinal disorders, in particular the treatment of spinal metastases or other degenerative, traumatic and/or congenital issues, is via corpectomy or vertebrectomy, desirably in combination with surgical arthrodesis of the spine. This can be accomplished by removing all or part of a vertebral body and the adjacent intervertebral disks, and replacing the removed tissues with implant(s) and/or bone and immobilizing the spine to allow the eventual fusion or growth of the bone across the treated space to connect the adjoining vertebral bodies together. The stabilization of the vertebra to allow fusion is generally assisted by the surgically implanted device(s) to hold the vertebral bodies in proper alignment and allow the bone to heal, much like placing a cast on a fractured bone. Such techniques have been effectively used to treat the above-described conditions and in most cases are effective at stabilizing the treated spinal region, concurrently reducing the patient's pain and preventing neurological loss of function.

As with many spinal disorders, there is need for further improvement in the devices, systems and surgical methods associated with such treatment. The present subject matter is such improvement. The present subject matter will now be described with reference to the drawings, wherein like reference numerals are used to refer to like elements throughout. It is to be appreciated that the various drawings are not necessarily drawn to scale from one figure to another nor inside a given figure, and in particular that the size of the components are arbitrarily drawn for facilitating the understanding of the drawings. In the following description, for purposes of explanation, numerous specific details are set forth in order to provide a thorough understanding of the present subject matter. It may be evident, however, that the present subject matter can be practiced without these specific details. Additionally, other embodiments of the subject matter are possible and the subject matter is capable of being practiced and carried out in ways other than as described. The terminology and phraseology used in describing the subject matter is employed for the purpose of promoting an understanding of the subject matter and should not be taken as limiting.

The implant devices and/or any portions or combination of portions thereof, such as those described and illustrated herein, can be constructed from radiopaque or radiolucent materials, other materials or combinations of such materials. Radiolucent materials can include, but are not limited to, polymers, carbon composites, fiber-reinforced polymers, plastics, combinations thereof and the like. One example of a radiolucent material that can be used with the present subject matter is PEEK-OPTIMA® polymer (commercially available from Invibio Inc., Greenville, S.C., USA). The PEEK-OPTIMA® polymer is a polyaromatic semicrystalline thermoplastic known generically as polyetheretherketone. The PEEK-OPTIMA® polymer is a biocompatible and inert material. Radiopaque materials are traditionally used to construct devices for use in the medical device industry. Radiopaque materials can include, but are not limited to, metal, aluminum, stainless steel, titanium, titanium alloys, cobalt chrome alloys, combinations thereof and the like.

In various embodiments, the implant devices and/or any portions or combination of portions thereof, such as those described and illustrated herein, can be constructed from an osteo-inductive and/or osteo-conductive material, such as Silicon Nitride. If desired, some portions of the implant may comprise an intermediate layer of a non-loading bearing material such as morselized bone graft and/or granular or powdered silicon nitride, with load bearing members such as titanium or PEEK positioned inside and/or outside of the non-load bearing members. For example, one or more bone facing surfaces of the disclosed plating devices could incorporate a surface coating of a silicon nitride material. The disclosed modular implants and/or “cage” structures can also allow for various combinations of materials to be integrated and implanted in a single cage. For example, an outer layer of silicon nitride to promote bony ingrowth may “cover” an inner layer of titanium that provides strength and/or support for the implant. A variety of such component materials could be employed, including metal, plastics and/or ceramics, including (but not limited to) PEEK, titanium, chrome cobalt, allograft, autograft or xenograft bone or other materials, solid Silicon Nitride and/or porous Silicon Nitride, as well as other materials well known in the art.

Radiolucent and/or other materials can be utilized to facilitate radiographic evaluation of fusion material or vertebrae near an implant device. For example, radiolucent materials permit x-rays to pass through the implant device or components thereof so that developed x-ray pictures provide more visibility of the fusion material and vertebrae without significant interference, such as imaging artifacts, caused by the implant device. Radiolucent materials can enable clear visualization through imaging techniques such as x-ray and computer tomography (CT), whereas traditional radiopaque metallic or alloy materials can generate imaging artifacts or scatter that may prevent a comprehensive inspection of the surrounding tissue, vertebra and fusion material. In order to address the general disadvantage that some radiolucent materials lack the strength of radiopaque materials, design modifications may be required to provide adequate structural integrity and durability to the implant device. For example, the thickness of portions of the implant device subject to stress and strain can be increased in order to add support and structural integrity. Thicker or bulkier construction can mitigate the stresses of vertebra migration and toggling of the bone fasteners that may cause the implant device to bend, crack or otherwise be damaged while in use.

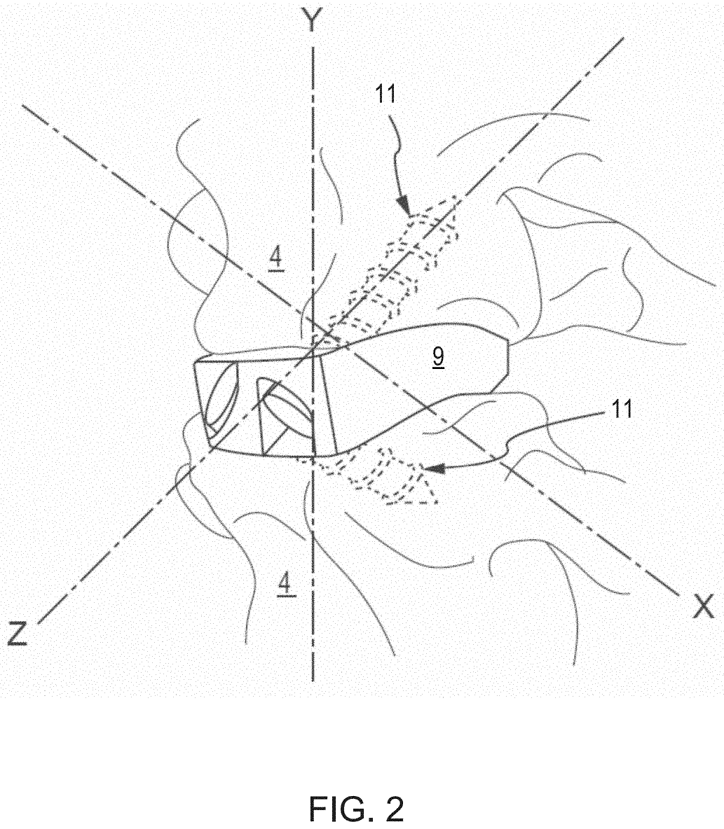

In various embodiment described herein, following a spinal surgical procedure, a medical professional may determine an appropriate size of an interbody device 9 (shown in FIG. 2) via one or more distractors and/or trials of various sizes. Each trial and/or distractor may be forcibly inserted between adjacent vertebrae 4. Upon determination of an appropriate size, one or more of an ACIF, ALIF, DLIF, PLIF, and/or TLIF may be performed by placing an appropriate interbody device 9 (such as, for example, a cage, a spacer, a block) between adjacent vertebrae 4 in the space formed by the removed degenerated disc 8. Placement of such interbody devices 9 within spinal column 2 may prevent spaces between adjacent vertebrae 4 from collapsing, thereby preventing adjacent vertebrae 4 from resting immediately on top of one another and inducing fracture of vertebra 4, impingement of the spinal cord, and/or pain. Additionally, such interbody devices 9 may facilitate fusion between adjacent vertebrae 4 by stabilizing adjacent vertebrae 4 relative to one another. Accordingly, as shown in FIG. 2, such interbody devices 9 often may include one or more bone screws 11 extending through interbody device 9 and into adjacent vertebrae 4.

Often, following the removal of the distractor and/or trial, a medical professional must prepare one or more bores or holes in a vertebra 4 intended to receive the bone screws 11. Such holes may be formed with the aid of a separate drill guide positioned proximate or abutting vertebra 4 and inserting a drill therethrough. Alternatively, such holes may be formed free hand, without the use of a drill guide. Further, since the spinal column 2 is subject to dynamic forces, often changing with each slight movement of the patient, such screw(s) 11 have a tendency to back out (for example, unscrew) and/or dislodge from interbody device 9, thereby limiting interbody device's 9 ability to stabilize adjacent vertebrae 4, and consequently, promote fusion. Additionally, if screw(s) 11 back out and/or dislodge from the interbody device 9, they may inadvertently contact, damage, and/or irritate surrounding tissue. Further, interbody device 9 is commonly comprised of a radiopaque material so as to be visible in situ via x-ray and other similar imaging modalities.

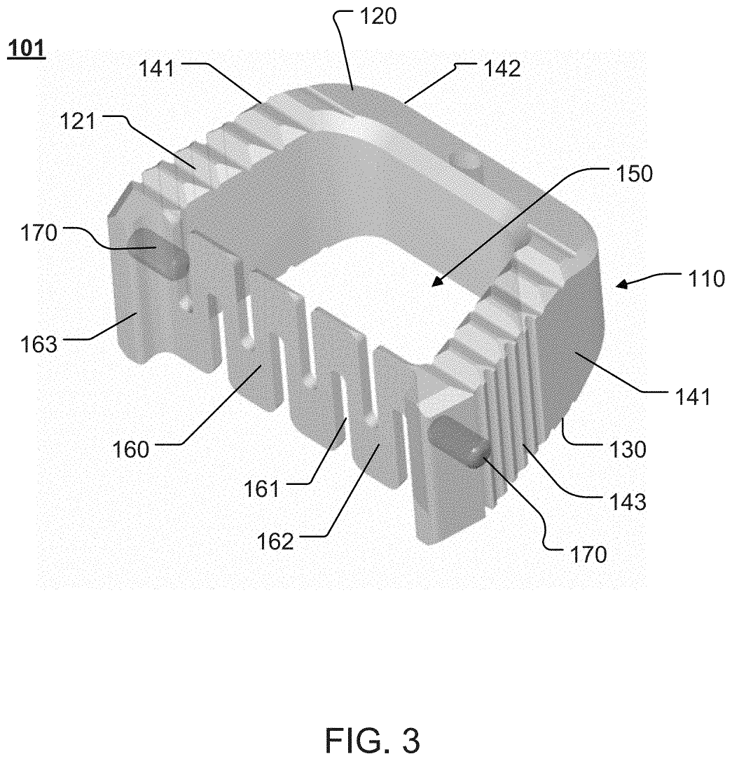

FIG. 3 illustrates one exemplary embodiment of a cage (or interbody) device 101 that can be constructed according to the principles of the disclosure. The cage 101 may include one or more features such as anti-migration and/or anchoring features, anti-rotation features, insertion tool features, reduced profile keel features, and the like. The cage 101 has a cage body 110 that may be formed as a single piece, or that may be assembled from multiple pieces. The cage body 110 may have a trapezoidal shape for proper anterior placement. The cage 101 may be made of one or more materials, including, for example, metal (e.g., titanium), metal alloy (e.g., titanium alloy), plastic, ceramic, elastomers, carbon fiber reinforced polymers, polyetheretherketone (PEEK), tricalcium phosphate, hyroxyaptaite, or the like, or any combination thereof. The cage 101 may have any shape, including, for example, a trapezoid, a square, a rectangle, a circle, an ellipse, a semicircle, or the like, or any combination of the foregoing, that may be implanted, for example, between a pair of adjacent vertebrae.

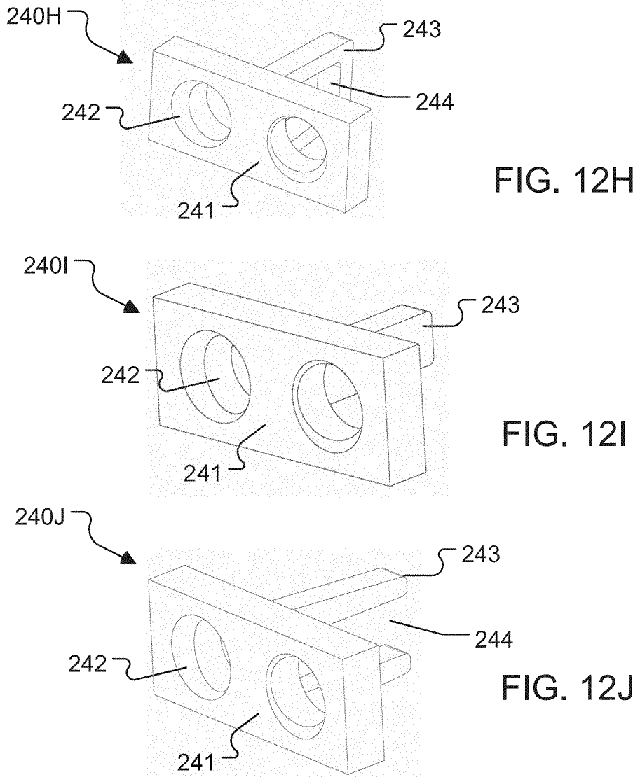

Referring to FIG. 3, the cage body 110 includes a pair of sagittal (or side) walls 141 and an aft-wall 142. The cage 101 may further include a fore-wall 160. The sagittal walls 141, aft-wall 142 and fore-wall 160 may form a chamber 150 that may receive and hold autologous bone, allograft bone, xenograft bone, bone graft material, osteoinductive material, blood, tissue, or the like. The chamber 150 may have a large graft area to provide generous biological coverage. The inner surfaces of the sagittal walls 141, aft-wall 142 and/or fore-wall 160 may have a smooth surface or a pattern that may help in holding, for example, a bone graft material in the chamber 150, such as, for example, a roughened surface, or a pattern that increases the coefficient of friction with respect to the bone graft material in the chamber 150. One or more of the outer surfaces of the side walls 141 and/or aft-wall 142 may include a grip interface 143. The grip interface 143 may have a pattern (e.g., teeth, serrations, protrusions, or the like) that increases gripability and improves handling by, for example, a surgeon's hand or instrument to securely grasp and hold the cage body 110 during implanting. The grip interface 143 may be configured to contact and engage a grip interface provided on an interbody device (for example, grip interface 2432 on the interbody device 240, shown in FIG. 13A), so as to secure the cage 101 to the interbody device.

The cage 101 may have a first surface 120 and a second surface 130. The first surface 120 may include a plurality of bone interface members 121, such as, for example, teeth, serrations, protrusions, which may have a shape that is, e.g., triangular, pyramidal, conical, semispherical, rectangular, cylindrical, diamond, elliptical, and/or irregular shapes, or the like. The first and second surfaces 120, 130 may have an aggressive pattern formed by the bone interface members 121 to resist expulsion. The first and second surfaces 120, 130, may be substantially the same or different. For instance, the first surface may include bone interface members 121 that have, for example, a pyramidal pattern and the second surface may include bone interface members (not shown) that have, for example, a pyramidal pattern and/or a semi-spherical pattern. The bone interface members 121 engage with the bony surface of vertebral bodies in or near the treated area. The bone interface members 121 may be formed integrally with the surface 120 (or 130) and may vary in profile, distribution, size, and number. The configuration of the surface 120 (or 130), including bone interface members 121, should be sufficient to securely hold the cage 101 in the treated area after surgery while the treated area heals and undergoes fusion.

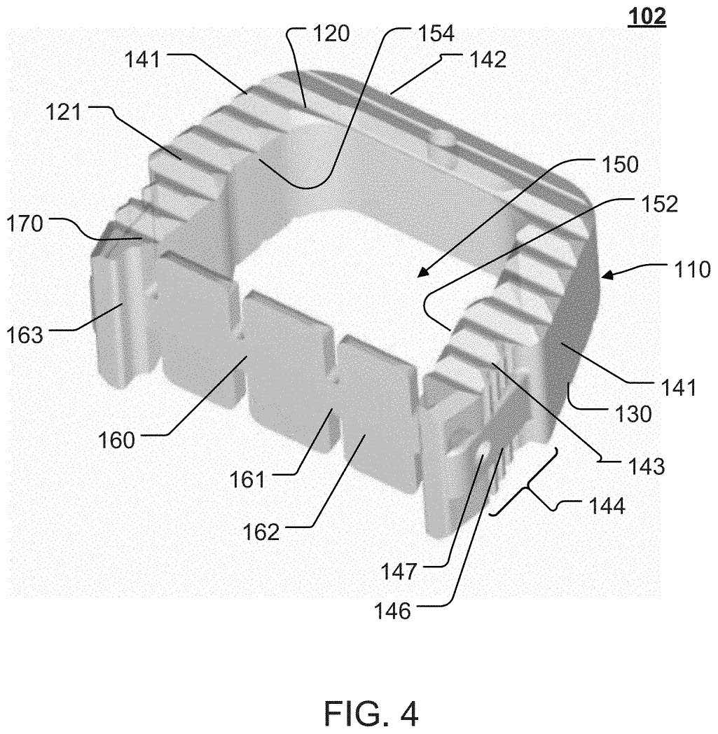

The fore-wall 160 may include a wall membrane 162, as seen in FIG. 3. The fore-wall 160 may include one or more slits 161. The slits 161 may be formed in one or both of the superior or inferior directions (that is, in the directions normal to the surfaces 120, 130). If the slits 161 are formed in both the superior and inferior directions, then the slits 161 may be offset from each other, so as to form a snake-like pattern (shown in FIG. 3). Alternatively (or additionally), the slits 161 may be formed so as to align with each other in the superior (or cranial) and inferior (or caudal) directions (shown in FIG. 4). The wall membrane 162 may be made of a thin, flexible and/or malleable material. The wall membrane 162 may be made of an elastic memory material that resumes a default shape absent an external force, such as, for example, the default shape seen in FIG. 3 (or FIG. 4). The material may include titanium, titanium alloy, PEEK, or the like. The forewall 160 may be constructed so as to be easily poked, breached, bent or penetrated by, for example, a hole preparation instrument (not shown), a bone fastener, or the like.

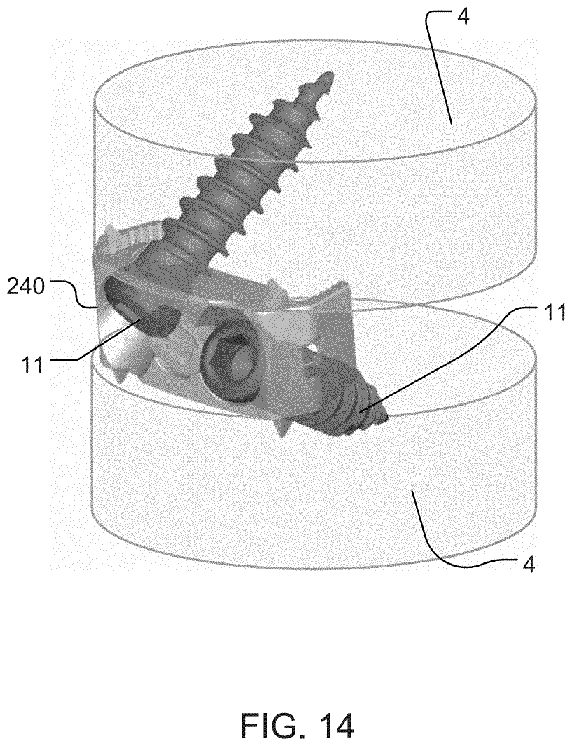

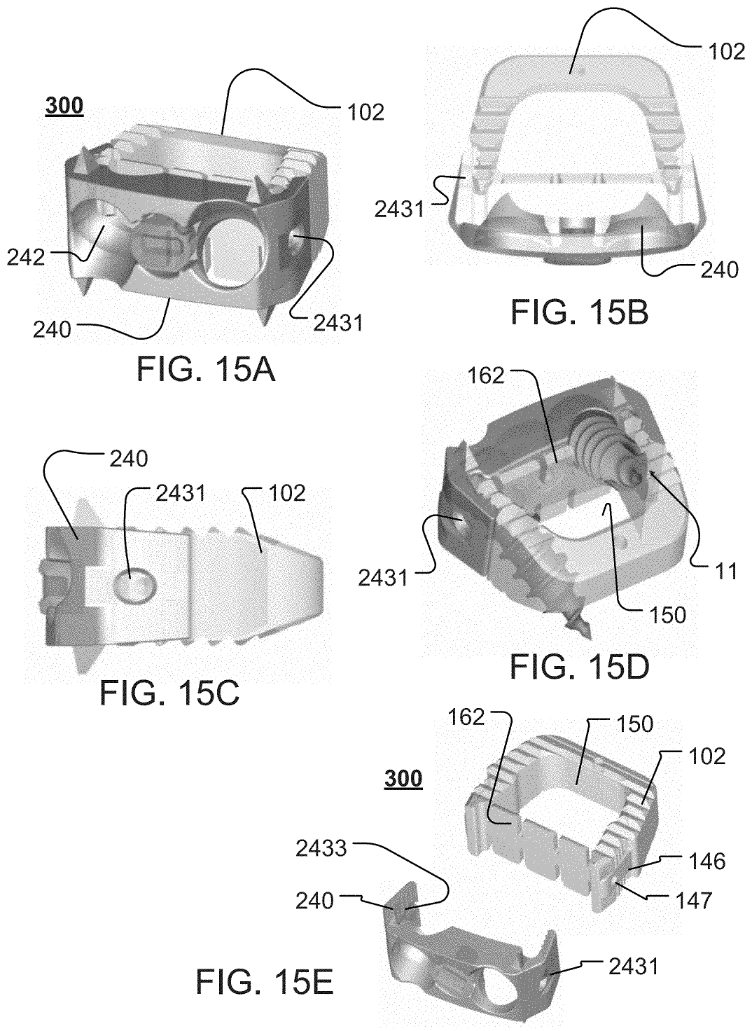

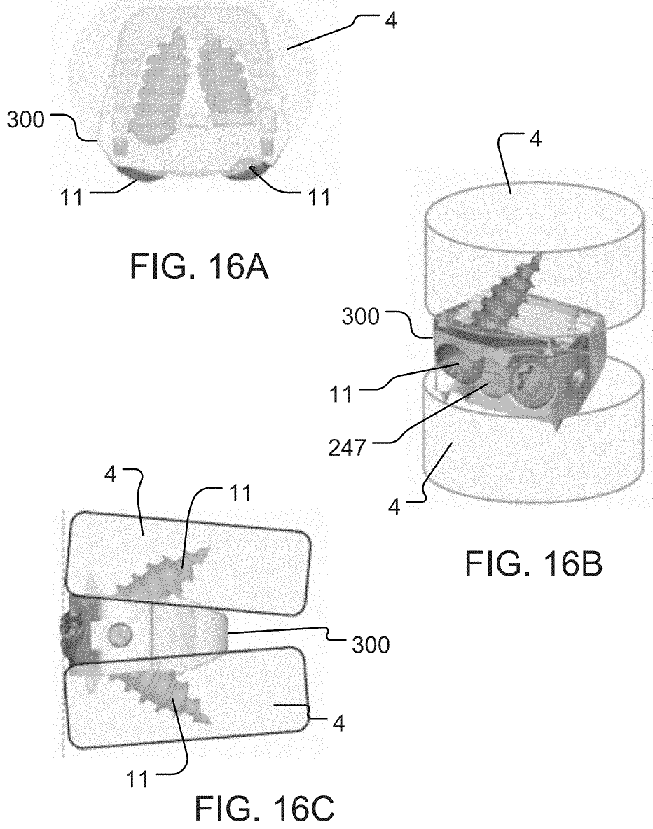

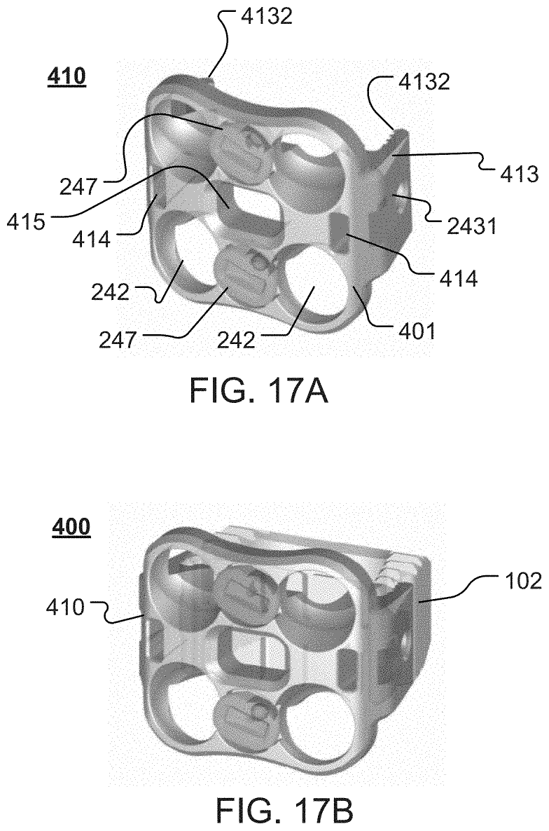

The wall membrane 162 may function to deflect and guide a bone fastener 11 to an anchoring position in the adjacent bone structure, as seen in FIGS. 15D and 16B, thereby facilitating easier and better positioning of the bone fastener 11 during installation of an interbody system (e.g., interbody system 300 or 400, shown in FIGS. 15A-15E and 17B-17E). For instance, referring to FIGS. 15A and 15D, as a bone fastener 11 is inserted through an aperture 242, the distal end of the fastener 11 may contact the wall membrane 162 and, as the fastener 11 is moved toward the wall membrane 162, the upper (or lower) portion of the wall membrane may bend and deflect the fastener 11 upward (or downward) toward the anchor site on the adjacent vertebra 4 (shown in FIGS. 16B and 16C).

Further, when a graft material is located in the graft chamber 150, deflection of the bone fastener(s) 11 will result in portion(s) of the wall membrane 162 moving into the graft chamber 150 and reducing the space in the chamber 150, thereby, forcing graft material upward and/or downward out of the graft chamber 150 and into the spaces surrounding the cage 101, including packing the graft material into the area between the cage 101 and adjacent vertebrae 4 to better promote bone growth.

The cage 101 may include one or more radiopaque elements 170 to assist with alignment, positioning or placement of the cage 101 in a treated area. The radiopaque element(s) 170 may include, for example, a radiopaque tantalum bead, or the like. The cage 101 may be provided with one radiopaque element 170 at each of three corners of the cage 101 to facilitate radiographic implant positioning.

The cage 101 may include one or more plate interfaces 163. The plate interface 163 may be integrally formed with the cage body 110. The plate interface 163 may be constructed as an extension of the side wall 141. The plate interface 163 may be configured to correspond to and mate with (or engage) a corresponding cage interface (e.g., cage interface 2463, shown in FIG. 13C) provided on an interbody device (e.g., interbody device 240 shown in FIGS. 13A-13C). When mated to the cage interface, the plate interface 163 may provide a secure and snug fit, so as to properly align the interbody device and cage 101 with respect to each other. The plate interface 163 and cage interface (e.g., cage interface 2463, shown in FIG. 13C) may be constructed as a tongue and groove configuration, with one of the interfaces being formed as the tongue portion and the other interface being formed as the groove portion.

FIG. 4 illustrates a perspective view of another example of a cage (or interbody) device 102, constructed according to the principles of the disclosure. The cage 102 includes a cage body 110. The cage body 110 includes a pair of sagittal (or side) walls 141 and the aft-wall 142. The cage 102 further includes the fore-wall 160. The side walls 141 may include a pair of recessed wall portions 144 located near the forewall 160. The inner surfaces of the side walls 141, the aft-wall 142 and fore-wall 160 form the graft chamber 150. The cage 102 may be made of one or more materials, including, for example, metal (e.g., titanium), metal alloy (e.g., titanium alloy), plastic, ceramic, elastomers, carbon fiber reinforced polymers, polyetheretherketone (PEEK), tricalcium phosphate, hyroxyaptaite, or the like, or any combination thereof.

The graft chamber 150 may include a chamber-width portion 152 and a chamber-width portion 154. The width the of the chamber-width portion 152 may be less than the width of the chamber-width portion 154. Alternatively, the width of the chamber-width portion 152 may be equal to or greater than the width of the chamber width portion 154. The chamber-width portion 152 may have a width formed between opposing inner wall surfaces of the side walls 141 by the inner wall surface of a plate interface region 144 on each of the side walls 141.

The plate interface region 144 of the side wall 141 may include the grip interface 143 and a plate guide 146. The wall plate interface region 144 may include a plate engager 147.

The plate guide 146 may be formed as, for example, a longitudinal track along the longitudinal axis of the side wall 141. The plate guide 146 may be configured to engage a corresponding cage guide (e.g., cage guide 2433, shown in FIGS. 13A, 13C) provided on an interbody device (e.g., interbody device 240, shown in FIGS. 13A, 13C), so as to facilitate proper positioning and alignment of the cage 102 with respect to the interbody device (e.g., interbody device 240 shown in FIGS. 13A-13C or interbody device 410 shown in FIGS. 17A-17E).

The plate engager 147 may be formed as an aperture (e.g., a semi-spherical recess, a dented-in portion, an opening that extends from the outer surface to the inner surface of the wall 141, or the like) or as a protrusion (e.g., a semi-spherical bump, or the like). The plate engager 147 may be positioned and configured to align with a cage engager on an interbody device (e.g., cage engager 2431 on interbody device 240, shown in FIG. 13A).

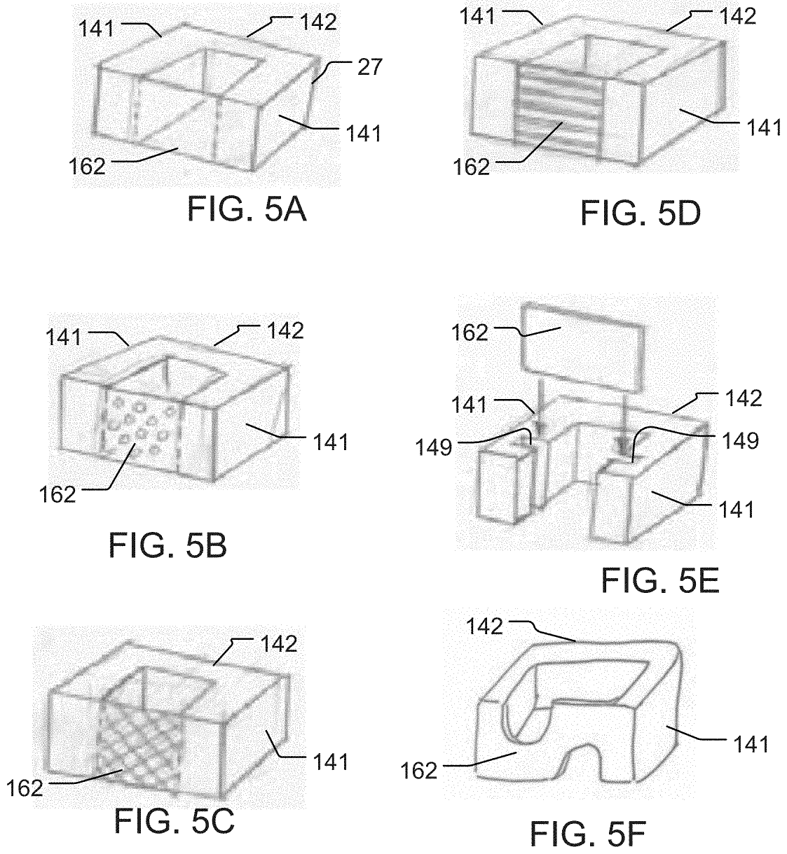

As seen in FIGS. 3 and 4, the wall membrane 162 may be formed integrally as part of the cage, or the wall member 162 may be attached to ends of the walls 140 (shown in FIGS. 5A-5E).

FIGS. 5A-5F illustrate examples of cages, with various embodiments optionally including different wall membranes 162, constructed according to the principles of the disclosure. As seen, FIG. 5A illustrates an example with the wall member 162 formed as a thin sheet that may be integrated with a cage body 27; FIG. 5B illustrates an example of the wall membrane 162 formed as a thin mesh, which may be integrated with or attached to the cage body 27; FIG. 5C illustrates an example of the wall membrane 162 formed as a thin screen, which may be integrated with or attached to the cage body 27; FIG. 5D illustrates an example of the wall membrane 162 that may be formed as a beams screen, which may be integrated with or attached to the cage body 27; FIG. 5E illustrates an example of attaching the wall membrane 162 by aligning and inserting ends of the wall member 162 into corresponding receiver tracks 149 that may be formed in the inner sides of the walls 141; and, FIG. 5F illustrates an example where the wall membrane 162 does not interact with bone fastener(s). The cage body 27 may be substantially the same as the cage body 110 (shown in FIG. 3 or 4).

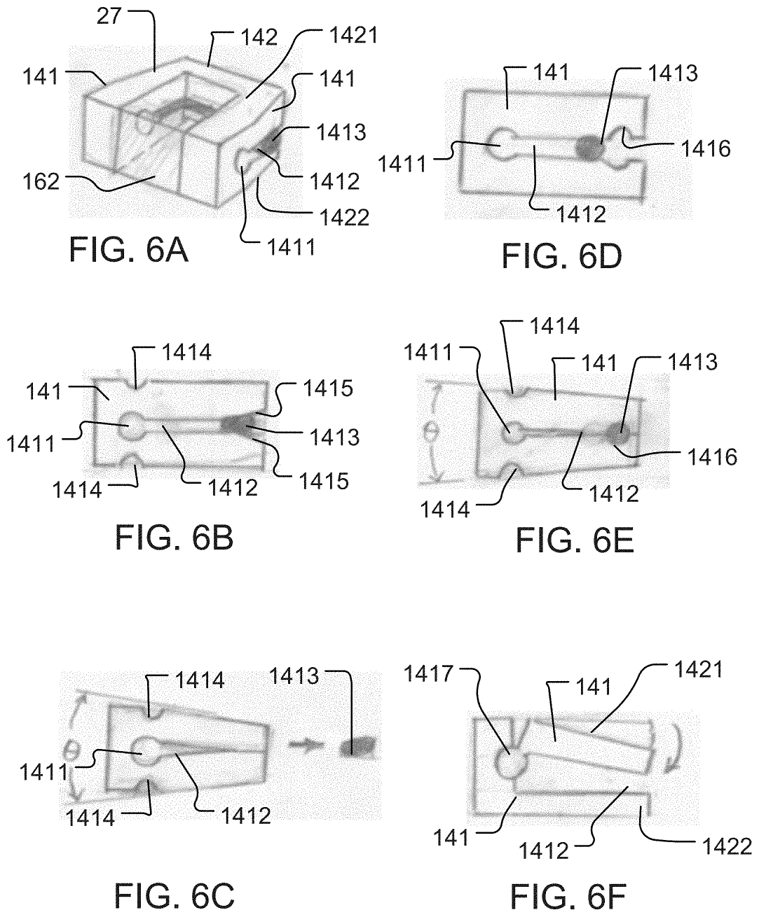

FIGS. 6A-6F illustrate further examples of cages, constructed according to the principles of the disclosure, which may be used to reduce inventory by providing a cage that may adjust angle of lordosis. As seen in the illustrations, the cage may include the cage body 27 with an angle adjustment system, which may include an angle adjustment slit 1412 formed longitudinally in the side walls 141 and aft-wall 142, and an angle adjustment insert 1413 (shown in FIGS. 6A-6E), or an angle adjustment hinge 1417 (shown in FIG. 6F). The cage body 27 may be made of a material, such as, for example, a shape memory material that reverts to a particular configuration in the absence of any external force (shown in FIG. 6C).

Referring to FIG. 6A, the cage body 27 may be formed with an angle adjustment slit 1412 in each of the side walls 141 and the aft-wall 142. The angle adjustment slit 1412 may be formed longitudinally along the longitudinal axis of the side wall 141 and the aft-wall 142, thereby separating the cage body 27 into a superior cage body portion 1421 and an inferior cage body portion 1422. The end portions of the superior cage body portion 1421 and the inferior cage body portion 1422 that are formed by the aft-wall 142 are configured to move toward or away from each other, thereby allowing for adjustment of angle of lordosis. The adjustment angle 8 (shown in FIG. 6E) may be defined and adjusted by, for example, inserting (or removing) the adjustment insert 1413 in one or both of the angle adjustment slits 1412. The adjustment angle may be varied and set by moving (e.g., incrementally) the adjustment insert 1413 along the angle adjustment slit(s) 1412.

The angle adjustment slit(s) 1412 may have a substantially uniform width along its entire length, or the width may vary along the length of the angle adjustment slit 1412 (e.g., increasing or decreasing). The adjustment insert 1413 may include, for example, a rod, a block, or any other shape without departing from the scope or spirit of the disclosure. The diameter or height of the adjustment insert 1413 may vary to provide varying angles of adjustment. The length of the adjustment insert 1413 may vary depending on the dimensions of the cage body 27. The cage body 27 may include a fulcrum aperture 1411, which may provide added flexibility to the cage body 27 with angle adjustment slits 1412. The fulcrum aperture 1411 may have a diameter (or width) that is greater than the width of the portion of the angle adjustment slit 1412 nearest to the fulcrum aperture 1411.

FIG. 6B shows the cage body 27 provided with flexing cutouts 1414 above and/or below the fulcrum aperture 1411. The flexing cutouts 1414 are formed to provide added flexibility to the cage body 27 with respect to the cage body portions 1421, 1422. The portion of the adjustment slit 1412 in the aft-wall 142 may include tapered wall portions 1415, so as to facilitate easier installation of the adjustment insert 1413 into the adjustment slit 1412.

FIG. 6C shows the cage body 27 with the adjustment insert 1413 removed from the adjustment slit 1412. In this example, the cage body 27 is formed of a memory-form material that reverts to a maximum adjustment angle 8 in the absence of an external force. The angle 8 may be adjusted by inserting the adjusting insert 1413 into the adjustment slit 1412. In this regard, the cage body 27 may include the tapered wall portions 1415 so as to facilitate easier insertion of the adjusting insert 1413 into the adjustment slit 1412.

FIGS. 6D and 6E illustrate examples of the cage body 27 provided with an insert lock 1416. As seen, the insert lock 1416 may be a notch, such as, for example, a notch formed longitudinally along the adjustment slit in the aft-wall 142. The notch may be formed longitudinally in the aft-wall 142 and/or transversely across the side wall(s) 141 (i.e., across the width of one or both side walls 141). The notch may have a shape such as, for example, a semi-circle, a square, a rectangle, or the like. The notch may be formed in either or both of the superior and inferior body portions 1421, 1422. The insert lock 1416 may have a height (or diameter) that is substantially equal to the height (or diameter) of the adjustment insert 1413. Alternatively, the height (or diameter) of the insert lock 1416 may be greater or less than the height (diameter) of the adjustment insert 1413. The insert lock 1416 may function to securely hold the adjustment insert 1413, so as to provide a predetermined adjustment angle 8. Although not shown, the adjustment slit 1412 may be provided with a plurality of insert locks 1416 along the length of the slit, each of which may be formed to provide a different, predetermined adjustment angle θ, which may be determined by the location of the insert lock 1416 along the adjustment slit, the height (or diameter) of the insert lock 1412, or the shape of the insert lock 1412.

FIG. 6F shows an example of a cage body 27 provided with a hinge 1417. As seen, the superior cage body portion 1421 and the inferior cage body portion 1422 may be connected by a hinge 1417 provided in the cage body 27 at the end opposite the aftwall 142. The hinge 1417 is configured to permit one or both of the cage body portions 1421, 1422 to pivot toward or away from each other, thereby adjusting the angle θ.

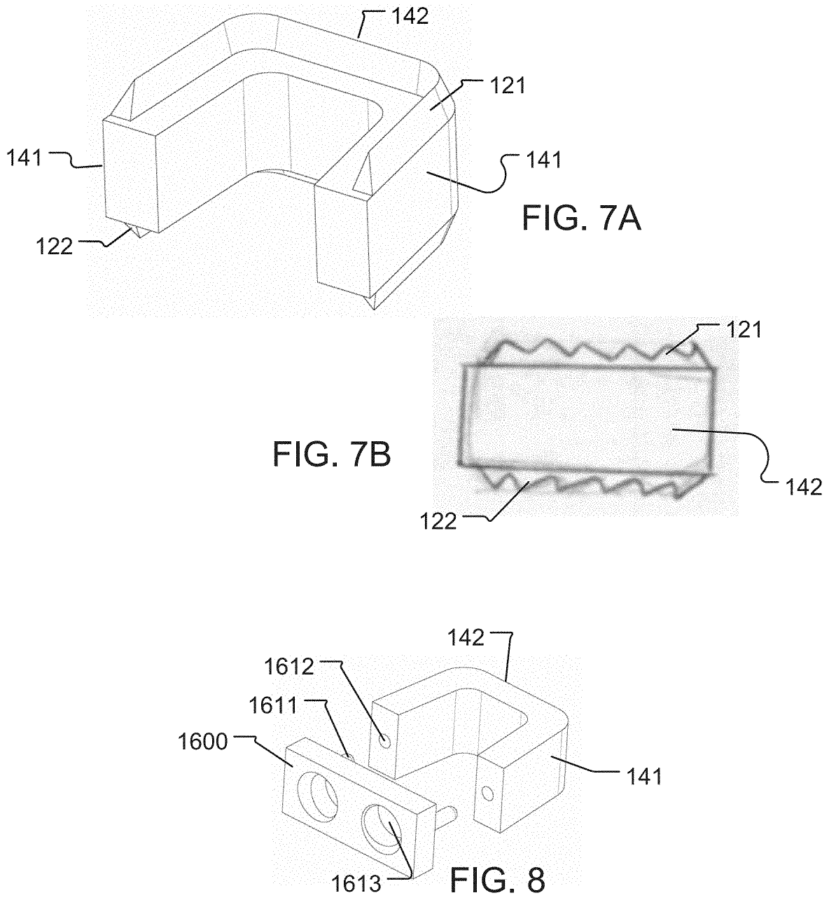

FIGS. 7A-7B illustrate different views of a cage body provided with a pair of anchor rims 121, 122. Although shown with two anchor rims 121, 122, the cage body may be provided with only a single anchor rim 121 (or 122) provided on one of the superior or inferior surfaces of the cage body. Alternatively (or additionally), a plurality of anchor rims 121 (or 122) may be provided on the same surface of the cage body. The anchor rim 121 (or 122) may be continuous (shown in FIG. 7A) or discontinuous (not shown, such as, e.g., in one or more segments). The anchor rim 121 (or 122) may have a consistent geometry (shown in FIG. 7A) or have a variable geometry (shown in FIG. 7B). The geometry may be, for example, a serrated pattern, a saw-tooth pattern, or any other pattern that may aggressively contact and engage boney matter.

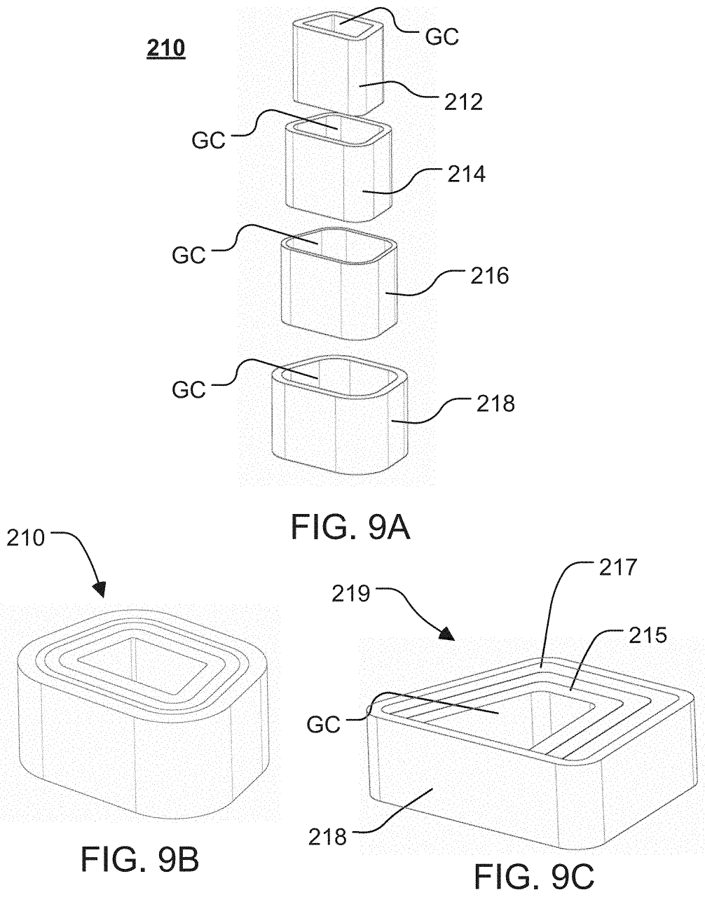

FIG. 8 illustrates an example of an interbody system, constructed according to the principles of the disclosure. The interbody system may include the cage body 27 and an interbody (or plate) device 1600. The interbody system may further include a wall membrane 162 (not shown), which may be positioned at, for example, the anterior portion of the cage body 27, and proximate the inner wall surface of the interbody device 1600. The interbody device 1600 may be attached to the cage body 27 by an attachment mechanism, such as, for example, a bonding or adhesive material, a pressure fit, tongue and groove, spring clamp, joining screws, or the like.

The interbody system of FIG. 8 is one example of a pressure fit attachment mechanism, which may include one or more receivers (e.g., openings) 1612 provided on the cage body 27, and corresponding one or more protrusions 1611 on the interbody device 1600. Alternatively, the one or more receivers 1612 may be provided on the interbody device 1600 and the corresponding one or more protrusions 1611 may be provided on the cage body 27. As seen, the receivers 1612 may be formed to align with and securely receive the corresponding protrusions 1611 when the interbody device 1600 and cage body 27 are pressed toward each other, so as to securely fasten the interbody device 1600 to the cage body 27.

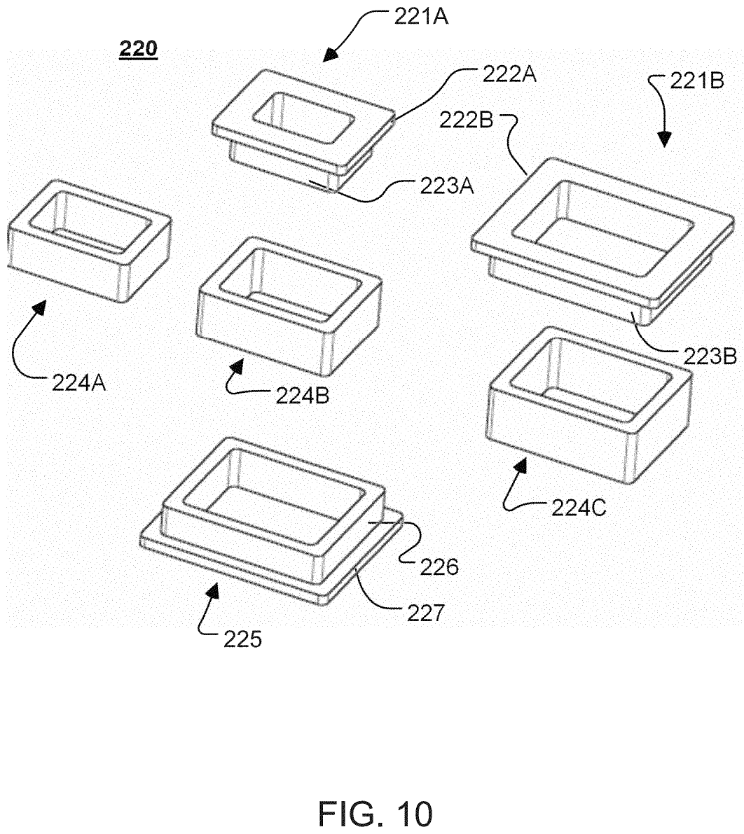

FIGS. 9A-9C illustrate examples of a modular cage system 210, constructed according to the principles of the disclosure. The shell-in-shell configuration of the modular cage system 210 can be used to minimize inventory of parts. The modular cage system 210 provides an adjustable footprint, wherein a closed loop geometry may be implemented (shown in FIGS. 9A and 9B), an open loop geometry may be implemented (not shown), or a hybrid closed-open loop geometry may be implemented (shown in FIG. 9C).

Referring to FIGS. 9A-9B, the modular cage system 210 may comprise a plurality of closed loop cage bodies 212, 214, 216, 218. Each of the cage bodies 212, 214, 216, 218, may have substantially the same shape and varying (e.g., increasing or decreasing) size (e.g., height, width, length, surface angle (e.g., angle of superior surface along posterior-anterior and/or lateral directions of cage body, and/or angle of inferior surface along posterior-anterior and/or lateral directions of cage body)), so that the cage bodies may be nested together to form a unitary configuration of the modular cage system 210, as seen in FIG. 9B, by nesting one inside another. One or more of the cage bodies 212, 214, 216, 218 may have a different shape and/or size than the other cage bodies. The cage bodies may be selected and nested together to form a cage system 210 that matches the size, shape, contours, etc. of the adjacent vertebrae surfaces. Each of the cage bodies 212, 214, 216, 218 may be made of a single material or combination of various materials for, for example, radio-opaque and/or strength effects. The cage bodies 212, 214, 216, 218 may be made of the same or different materials. The modular cage system 210 may include, for example, two, three, four, or more cage bodies.

The cage bodies 212, 214, 216, 218 may optionally each have a graft chamber GC, whose dimensions and position may be varied by varying the thicknesses and/or shapes of the walls of the respective cage body. For instance, by making one of the four walls of the cage body 212 much thicker than the other three walls, the center of the graft chamber GC may be shifted away from the thicker wall. Further, by altering the inner contours of the walls of a cage body, the shape of the graft chamber GC may be selectively determined. The outer contours of the walls of one or more of the cage bodies 212, 214, 216, 218 may be varied to form cage bodies based on the particular anatomy of a patient.

Referring to FIG. 9C, a hybrid modular cage system 219 comprises a pair of open loop cage bodies 215,217 nested in the closed loop cage body 218. The open loop cage bodies 215, 217 may each be formed with three walls, as seen in the illustration. Each of the cage bodies 215, 217, may have substantially the same shape and increasing (or decreasing) size, so that the cage bodies may be nested together to form a unitary configuration of the modular cage system 219, as seen in FIG. 9C, by nesting one inside another. The cage body 218 may have substantially the same (or different) shape as the open loop cage body 215 and/or 217, so as to receive and hold each of the cage bodies 215,217 in the graft chamber GC of the cage body 218. One or more of the cage bodies 215, 217, 218 may have a different shape than the other cage bodies. Each of the cage bodies 215, 217, 218 may be made of a single material or combination of various materials for, for example, radio-opaque and/or strength effects. The cage bodies 215, 217, 218 may be made of the same or different materials.

One or more of the cage bodies 215,217 may be nested in the cage body 218 to modify the dimensions, position and/or shape of the graft chamber GC in the cage body 218. By selecting wall dimensions and shapes of each of the cage bodies 215, 217, and nesting the cage bodies 215, 217 in a predetermined direction, the dimensions, position and/or shape of the graft chamber GC may be selectively determined. The predetermined direction may comprise, for example, the open end of the cage body 215 facing in the same or a different direction than the open end of the cage body 217. As seen in FIG. 9C, the open ends of the cage bodies 215, 217 may be positioned in the same direction, so as to position the center of the graft chamber GC toward the open end of the cage bodies 215, 217, when nested in the configuration seen in FIG. 9C. By making one of the walls of a cage body much thicker than the other three walls, the center of the graft chamber GC may be shifted away from the thicker wall. Further, by altering the inner contours of the walls of a cage body, the shape of the graft chamber GC may be selectively determined. The outer contours of the walls of one or more of the cage bodies 215, 217, 218 may be varied to form cage bodies based on the particular anatomy of a patient.

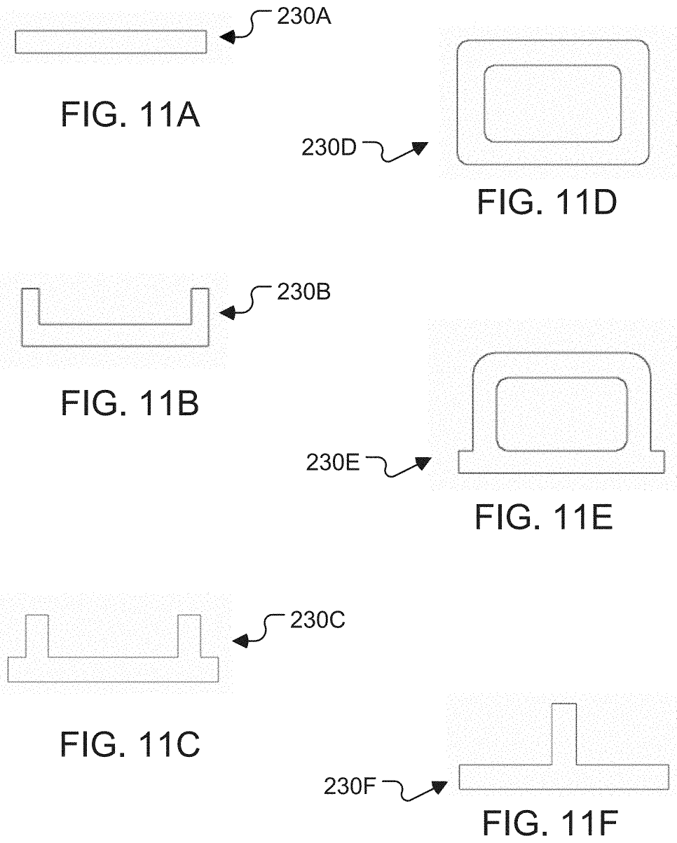

FIG. 10 illustrates an example of modular cage system 220, constructed according to the principles of the disclosure. The modular cage system 220 may comprise a plurality of closed loop cage bodies 224A, 224B, 224C. The modular cage system 220 may, instead, include all open loop cage bodies (not shown), or a hybrid system of open and closed loop cage bodies (not shown). The modular cage system 220 may further include one or more end caps 221A, 221B, 225. Each of the cage bodies 224A, 224B, 224C, may have substantially the same shape with varying (e.g., increasing or decreasing) size (e.g., height, width, length, surface angle (e.g., angle of superior surface along posterior-anterior and/or lateral directions of cage body, and/or angle of inferior surface along posterior-anterior and/or lateral directions of cage body)), as seen in FIG. 10, so that the cage bodies 224A, 224B, 224C may be interchangeably used with one or more of the end caps 221A, 221B, 225. One or more of the cage bodies 224A, 224B, 224C may have a different shape than the other cage bodies. Each of the cage bodies 224A, 224B, 224C and/or the end caps 221A, 221B, 225 may be made of a single material or combination of various materials for, for example, radio-opaque and/or strength effects. The cage bodies 224A, 224B, 224C and/or the end caps 221A, 221B, 225 may be made of the same or different materials.

The cage bodies 224A, 224B, 224C may have any shape that may be implemented in an application between vertebral bodies, as will be understood by those skilled in the art. For instance, the cage bodies 224A, 224B, 224C may have a trapezoidal shape, with the side walls tapered inward in the posterior direction (e.g., shape of cage body 102 shown in FIG. 4), or the shape of the cage bodies 224A, 224B, 224C may be a square, rectangular, elliptical, circular, semicircular, or the like. The end caps 221A, 221B, 225 may have a shape that matches the shape of the cage bodies 224A, 224B, 224C.

As seen in FIG. 10, the end caps 221A, 221B, 225 may each include an insert portion 223A, 223B, 226, respectively, and/or a rim portion 222A, 222B, 227, respectively. For instance, referring to the end cap 221A with the understanding that the description equally applies to the end caps 221B and 225, the end cap 221A includes an insert portion 223A that may be inserted into the opening of the cage body 224A (or 224B or 224C), and a rim portion 222A that may function as a stop and/or cap for the cage body 224A (or 224B or 224C). The thickness, size and/or shape of the wall portions that form the insert portion 223A may be predetermined so as to selectively determine the position, shape, and/or size of the graft chamber in the cage body 224A (or 224B or 224C). For instance, the walls of the insert portion 223A may be varied in terms of size and shape, including, for example, height, width, length, surface angles, so as to determine the shape, position and size of the graft chamber in the cage body 224A (or 224B or 224C) when the end cap 221A is attached to the cage body 224A.

Similarly, the thickness, size and/or shape of the rim portion 222A may be varied to, for example, match anatomical requirements for particular applications of the cage system. For instance the height of the walls that form the rim portion 222A may be decreased (or increased) in the posterior (or anterior) direction, so as to provide better fit in vertebral interbody applications. The rim portion 222A may be configured to contact and engage a vertebral body. In this regard, the surface of the rim portion 222A may be contoured to match the shape of the vertebral body. The surface may include bone interface members (e.g., bone interface members 121, shown in FIG. 3) that may be configured to aggressively grip against the bony surface of the adjacent vertebral body (e.g., vertebral body 4, shown in FIG. 2).

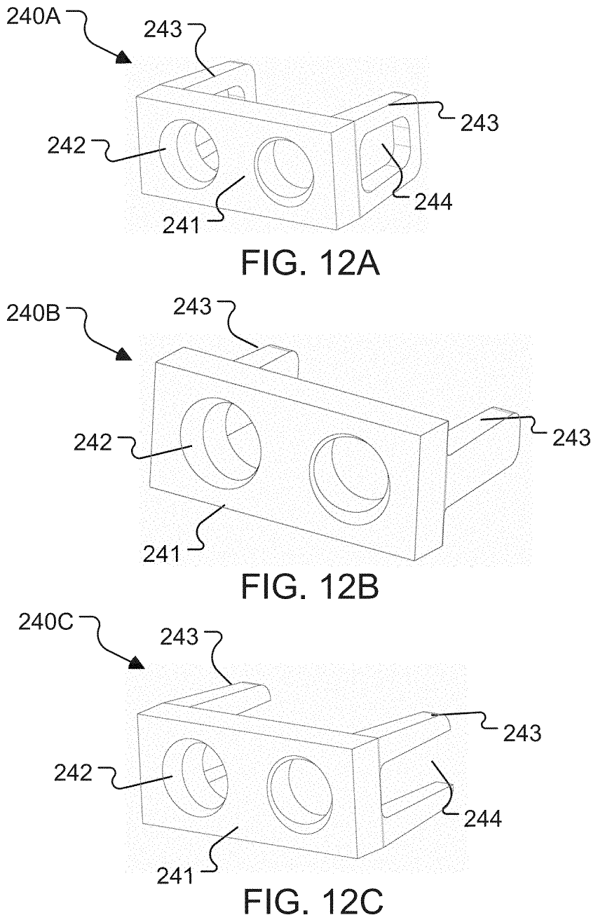

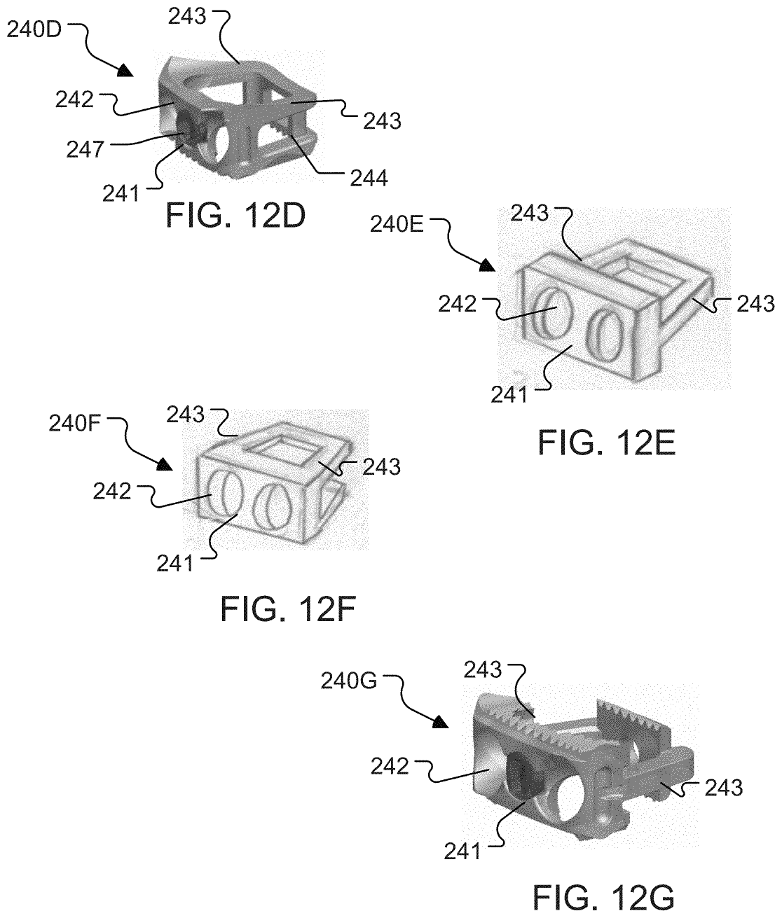

FIGS. 11A-11F show superior/cranial (or inferior/caudal) views of examples of interbody devices 230A-230F, respectively, constructed according to the principles of the disclosure. More specifically, FIG. 11A illustrates the superior or cranial (or inferior or caudal) view of interbody device 230A that may be substantially planar, having an anterior surface (not shown) and a posterior surface (not shown) with dimensions that are significantly greater than any one of the wall surfaces, including the superior (or inferior) surface (shown in FIG. 11A), and sagittal (or side) surfaces (not shown); FIG. 11B illustrates the superior (or inferior) view of interbody device 230B that has a C-shape (see also perspective views of examples of C-shape interbody devices in FIGS. 12A-12C) in the transverse plane; FIG. 11C illustrates the superior (or inferior) view of interbody device 230C that has a C-offset shape in the transverse plane; FIG. 11D illustrates the superior (or inferior) view of interbody device 230D that has a quadrilateral shape (see also perspective views of examples of trapezoid shape interbody devices in FIGS. 12D-12G) in the transverse plane; FIG. 11E illustrates the superior (or inferior) view of interbody device 230E that has a quadrilateral offset shape in the transverse plane; and FIG. 11F illustrates the superior (or inferior) view of an I-beam shape interbody device 230F in the transverse plane.

FIGS. 12A-12J illustrate perspective views of examples of interbody devices 240A-240J, respectively, constructed according to the principles of the disclosure. More specifically, FIGS. 12A-12C illustrate perspective views of examples of C-shape interbody devices 240A-240C, respectively; FIGS. 12D-12G illustrate perspective views of examples of transverse plane quadrilateral shape interbody devices 240D-240G, respectively; and FIGS. 12H-12J illustrate perspective views of examples of lateral I-beam shape interbody devices 240H-240J, respectively.

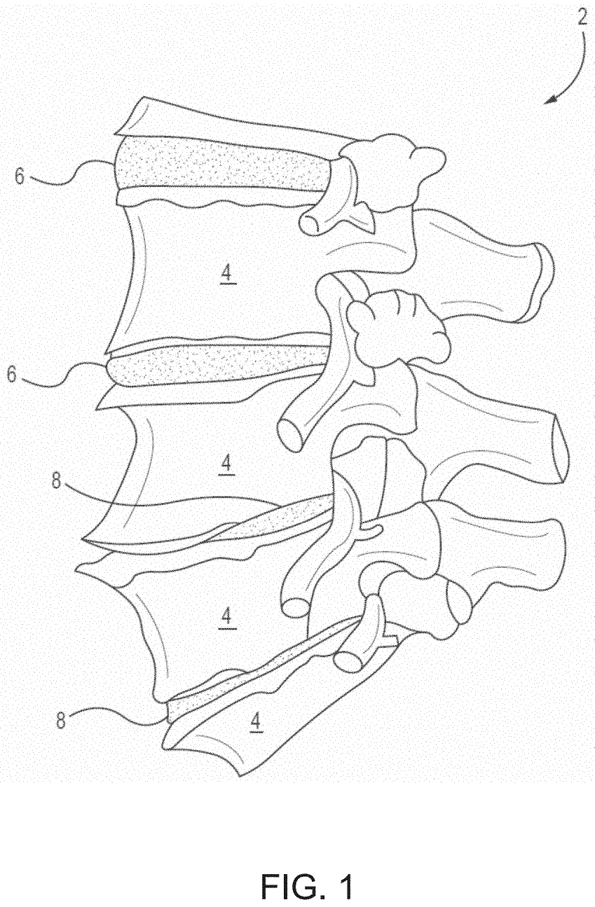

Referring to FIG. 12A, interbody device 240A may include a face 241 defining two apertures 242. The interbody device 240A may include a locking element 247, which is described in detail below. Various arrangements of interbody devices 240A may include one or more features configured to facilitate sagittal and/or coronal visibility. For example, a body or frame 243 of interbody device 240A may comprise a radiopaque material visible via x-ray or similar forms of imaging modalities. As such, frame 243 may enable accurate positioning and/or placement of interbody device 240A within and/or along spinal column 2 (shown in FIG. 1). Frame 243 may include any one or more features such as anti-migration and/or anchoring features, anti-rotation features, insertion tool features, reduced profile keel features, and the like. Additionally, frame 243 may define one or more openings and/or windows 244. Such windows 244 may remain empty and/or may be filled with radiolucent material such as tissue grafts as will be described in further detail below. Window(s) 244 may enable a medical professional to view and/or determine the level of post-operative fusion between interbody device 240A and patient bone and/or tissue. Frame 243 may define any appropriate arrangement, number, and configuration of window(s) 244. That is, as shown in FIG. 12A, for example, interbody device 240A may comprise a standalone device having an open cage, or the interbody device 240A may be used as a plate device and attached to a cage (not shown). As shown in FIG. 12A, frame 243 may include a single window 244 on each lateral side. Each window 243 may be generally quadrilateral (e.g., square, rectangular, or trapezoidal). In some arrangements, a radiolucent structure, such as a graft containment sheath, may be disposed along one or more portions of frame 243. Indeed, such graft containment sheaths may substantially fill or encompass window 244 of one or more sides of frame 243. Accordingly, when the interbody device 240A is placed between two adjacent vertebrae 4 (shown in FIG. 1) under X-ray vision, window 244 remains radiolucent such that fusion within and/or through window 244 may be observed. In another arrangement, the interbody device 240B may comprise a standalone device having an open cage as seen in FIG. 12B, or it may be combined with a cage body (not shown), such as, for example the cage body 10 shown in FIG. 62 of U.S. patent application Ser. No. 14/956,084, filed Dec. 1, 2015, titled “Intervertebral Implants and Related Systems and Methods,” the entire disclosure of which is incorporated herein by reference.

Referring to FIGS. 12A and 12C, the interbody device 240C may have a similar arrangement to the interbody device 240A, except that the window(s) 244 may be open on the posterior end, as seen in FIG. 12C.

Referring to FIGS. 12D and 12G, the interbody devices 240D and 240G may be substantially the same as the devices 10 shown in FIGS. 33 and 35, respectively, in U.S. patent application Ser. No. 14/956,084, filed Dec. 1, 2015, titled “Intervertebral Implants and Related Systems and Methods,” which has been incorporated herein by reference.

FIGS. 12E-12F examples of interbody devices 240E and 240F that have I-beam shape and a C-shape, respectively, in the sagittal plane. Both devices 240E and 240F have a closed trapezoidal shape frame 243 in the transverse plane.

FIGS. 12H-12J illustrate perspective views of examples of interbody devices 240H-240J that have an I-beam shape in the transverse plane, but varying arrangements in the sagittal plane. For instance, the interbody device 240H includes a frame 243 that has substantially quadrilateral (e.g., trapezoidal, rectangular, or the like) closed shape in the sagittal plane, which may include a window 244. The interbody device 2401 includes a frame 243 that is substantially a rectangular rod in both the transverse and sagittal planes. The interbody device 240J includes a frame 243 that has a C-shape in the sagittal plane, including an open-ended window 244.

FIGS. 13A-13C illustrate different views of an example of an interbody device 240 that is constructed according to the principles of the disclosure, including a perspective front or anterior view (FIG. 13A), a front or anterior view (FIG. 13B), and a perspective back or posterior view (FIG. 13C).

Referring to FIGS. 13A-13C, the interbody device 240 may include the anterior or front coronal face 241 and a posterior or back coronal face 248 (shown in FIG. 13C) defining a plurality (e.g., two) apertures 242 therebetween. The interbody device 240 may include a locking element 247. The interbody device 240 may be formed as a single piece (not shown), or it may be assembled from two or more pieces, such as, for example the frame 243 and the locking element 247.

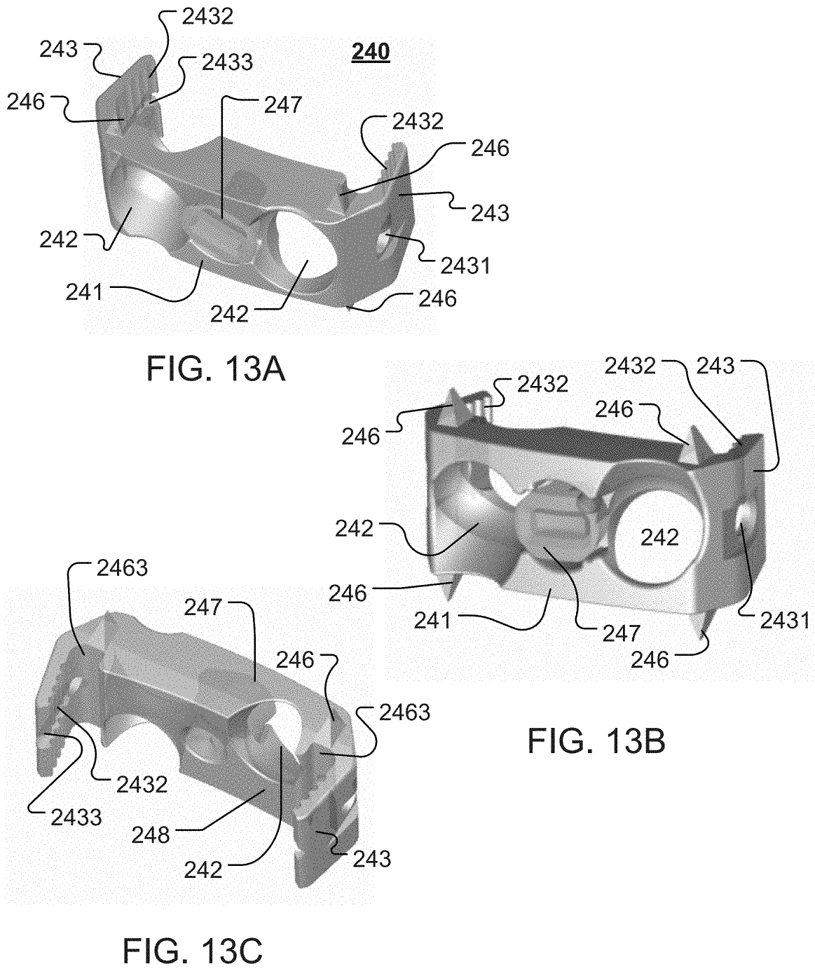

The aperture(s) 242 may have an angled opening so as to better guide a fastener 11 (shown in FIG. 14) as it is inserted in and through the aperture 242 into adjacent bone, thereby securing the fastener 11 in adjacent vertebra (shown in FIG.14) in, for example, an optimal angle for securing the interbody device 240 to the vertebrae 4.

The locking element 24 7 may be similar in construction and manner of use as described, for example, in FIGS. 3A-22D, 33, 35, 37, 39, 55, 58-65B, or 69A-78E and the corresponding text in U.S. patent application Ser. No. 14/956,084, filed Dec. 1, 2015, titled “Intervertebral Implants and Related Systems and Methods,” which has been incorporated herein by reference. Further, various arrangements of interbody devices 240 may include one or more features configured to facilitate sagittal and/or coronal visibility. For example, the body or frame 243 of interbody device 240 may comprise a radiopaque material visible via x-ray or similar forms of imaging modalities. As such, frame 243 may enable accurate positioning and/or placement of interbody device 240 within and/or along spinal column 2 (shown in FIG. 1). Frame 243 may include any one or more features such as anti-migration and/or anchoring features, anti-rotation features, insertion tool features, reduced profile keel features, and the like, as will be described in further detail below.

For instance, the frame 243 may include anti-migration and/or anchoring features 2432, 246. The features 2432 may be configured to contact and engage surface portions of, for example, a cage 102 (or 101) (shown in FIGS. 15A-15D) to secure the interbody device 240 to the cage 102. The features 2432 may be configured to assist in aligning and proper positioning of the interbody device 240 with respect to the cage 102 (or 101).

The anti-migration and/or anchoring features 246 may be located on upper and/or lower surfaces of the interbody device 240 that contact bone surface(s).

The features 2432 and/or 246 may comprise, for example, a pattern and/or texture that provides anti-migration and/or anchoring characteristics when implanted in the spine 2.

The features 2432 and/or 246 may comprise, e.g., teeth, serrations, protrusions (e.g., triangular, pyramidal, conical, semispherical, rectangular, cylindrical, diamond, elliptical, and/or irregular shapes, or the like), or the like.

The frame 243 may include a channel 2433, as seen in FIGS. 13A and 13C. The channel 2433 may be provided on one or both inner walls of the frame 243. The channel 2433 may be configured to receive, engage and guide a plate guide of, for example, a cage 102 (e.g., plate guide 146 of cage 102, shown in FIG. 4), thereby providing proper alignment and positioning of the interbody device 240 with respect to the cage 102 (e.g., as seen in FIGS. 15A-15D).

Alternatively, the frame 243 may include a guide element (not shown), such as, for example, the plate guide 146 (shown in FIG. 4), that is configured to be received, engaged, and guided by a channel (not shown) that may be provided in the cage body.

The interbody device 240 may further include an engager element 2431. The engager element 2431 may be positioned and configured to align with and engage a plate engager, such as, for example, the plate engager 147 (shown in FIG. 4) to secure the interbody device 240 into final position with respect to the cage body (e.g., shown in FIGS. 15A-15C). The engager element 2431 may be arranged as a male element such as a protrusion, or the like, provided on an inner wall of the frame 243, or a female element such as a recess provided in the inner wall of the frame 243, or an opening formed through the wall of the frame 243, or the like. In this regard, the plate engager (e.g., plate engager 147, shown in FIG. 4) may include an oppositely configured element that engages and mates with the engager element 2431 to secure the cage (e.g., cage 102 shown in FIGS. 15A-15D) to the interbody device 240.

Referring to FIGS. 13A-13C and 14 simultaneously, the interbody device 240 may be configured for use in, for example, anterior approach and discectomy applications. For instance, after a patient is positioned in a supine position on, for example, a radiolucent operating table, the surgical area cleaned, an incision made, muscle tissue and/or organs moved to the side(s), and other common surgical procedures carried out, a disc may be incised, removed, and the space prepared for implanting of an interbody device 240. The bone surfaces and edges on the adjacent vertebrae 4 may be carefully contoured, as appropriate.

Following a discectomy or other surgical procedure, a medical professional may determine an appropriate size of an interbody device 240 by selecting an appropriately dimensioned interbody device 240, which may be selectable based on, for example, height, width, depth, surface angle(s), and the like. Upon selecting the appropriate interbody device 240, one or more of an ACIF, ALIF, or the like may be performed by placing the interbody device 240 between adjacent vertebrae 4 in the space formed by the removed degenerated disc (shown in FIG. 14). One or more fasteners 11 may be installed using an instrument (not shown), such as, for example, a screw driver (not shown). The locking element 247 may then be turned or otherwise manipulated to secure the fasteners 11 in place, thereby preventing the fasteners 11 from loosening or withdrawing from the bone.

Placement of the interbody device 240 within spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, such interbody device 240 may facilitate fusion (e.g., bone to grow together) between adjacent vertebrae 4 by stabilizing adjacent vertebrae 4 relative to one another.

FIGS. 15A-15E illustrate different views of an interbody system 300 that includes the interbody device 240 and the cage 102 (or 101, shown in FIGS. 3 and 4). The interbody system 300 may be formed as a single structure (not shown), or it may be assembled from two or more pieces. It should be understood that the various implant component described herein, including screw or other fixation devices, plates, cages, modular components and/or the like may be utilized in a variety of combinations, depending upon the surgeon's preference and/or appropriate component compatibility. For example, the various interbody devices and/or plates may be assembled with the various disclosed cages and/or other components, if desired. Thus various combinations of all of the individual components disclosed herein are contemplated.

For instance, referring to FIG. 15E, the interbody system 300 may be assembled by attaching the interbody device 240 to the cage 102 (or 101). In this regard, the interbody 240 may be positioned so that the channels 2433 are aligned with corresponding plate guides 146 on the cage 102, as seen in FIG. 15E. The interbody device 240 and cage 102 may then be moved toward each other, with the plate guides 146 being received and guided by the respective channels 2433 as the cage 102 moves toward the back face 248 (shown in FIG. 13C) of the interbody device 240, until, for example, the engager element 2431 is aligned with and/or engages the plate engager 147, thereby securing the interbody device 240 to the cage 102 to form the interbody system 300.

FIGS. 16A-16C illustrate an example of implanting the interbody system 300 between adjacent vertebrae 4. More specifically, FIG. 16A illustrates a top or superior (or bottom or inferior) cross-sectional view of the spine 2 (shown in FIG. 1) with the interbody system 300 implanted; FIG. 16B shows a perspective anterior/coronal view of the implanted interbody system 300; and, FIG. 16C shows a sagittal view of the implanted system 300.

The interbody system 300 may be configured for use in, for example, anterior approach and discectomy applications. The interbody system 300 may be implanted between the vertebrae 4 in similar manner to that described above with reference to FIG. 14. That is, the patient may be positioned in a supine position on, for example, a radiolucent operating table, the surgical area cleaned, an incision made, muscle tissue and/or organs moved to the side(s), and other common surgical procedures carried out. A disc may then be incised, removed, and the space prepared for implanting of the interbody system 300. The bone surfaces and edges on the adjacent vertebrae 4 may be carefully contoured, as appropriate.

Prior to the surgery (based on preoperative imaging, for example) or during the surgical procedure, the medical professional may determine an appropriate size of the interbody system 300 by selecting an appropriately dimensioned interbody system 300, which may be selectable based on, for example, height, width, depth, surface angle(s), and the like. If the interbody device 240 and cage 102 are provided separately, the medical professional may select an interbody device 240 having appropriate dimensions (such as height, width, depth, surface angles, and the like) for the particular procedure and patient's anatomy, and the medical professional may similarly select a cage body 102 having appropriate dimensions (such as height, width, depth, surface angles, and the like) for the procedure and patient's anatomy. The medical professional may then assemble the interbody device 240 and cage 102 to form the interbody system 300, as shown in FIG. 15E. The medical professional may then place the interbody system 300 in the prepared space between the vertebrae 4.

Upon selecting the appropriate interbody system 300, one or more of an ACIF, ALIF, or the like may be performed by placing the interbody system 300 between adjacent vertebrae 4 in the space formed by the removed degenerated disc (shown in FIGS. 16B and 16C). One or more fasteners 11 may be installed using an instrument (not shown), such as, for example, a screw driver (not shown). As each fastener 11 is inserted through the aperture 242 and into contact with the wall membrane 162, the wall membrane 162 may bend and provide directional support against the skyping due to the springboard effect of wanting to back into its natural state, thereby urging the fastener 11 to the anchoring site. Simultaneously, due to the pushing of the wall membrane 162 into the graft chamber 150, the wall membrane may direct graft material from the graft chamber 150 to the areas surrounding the interbody system 300. After the fasteners 11 are implanted in their final positions in the anchoring sites, the locking element 247 may be turned or otherwise manipulated to secure the fastener(s) 11 in place, thereby preventing the fastener(s) 11 from backing out (e.g., unscrewing) and/or dislodging from the anchor site(s).

Placement of the interbody system 300 within spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, such interbody system 300 may facilitate fusion (e.g., bone to grow together) between adjacent vertebrae 4 by stabilizing adjacent vertebrae 4 relative to one another.

FIG. 17A illustrates another example of an interbody device 410. The interbody device 410 may include the anterior coronal (or front) face 401 and a posterior coronal (or back face, not shown) defining a plurality (e.g., four) apertures 242 therebetween. The interbody device 410 may include one or more (e.g., two) locking elements 247.

The interbody device 410 may include a window 415. The window 415 may provide access and/or visibility to the space behind the back face (not shown) of the interface device 410. The window 415 may remain empty and/or may be filled with radiolucent material such as tissue grafts. The window 415 may enable a medical professional to view and/or determine the level of post-operative fusion between interbody device 410 and patient bone and/or tissue. The window 415 may be generally quadrilateral (e.g., square, rectangular, or trapezoidal). In some arrangements, a radiolucent structure, such as a graft containment sheath, may be disposed over the window. Indeed, such graft containment sheaths may substantially fill or encompass the window 244. Accordingly, when the interbody device 410 is placed between two adjacent vertebrae 4 under X-ray vision, window 415 remains radiolucent such that fusion within and/or through window 415 may be observed.

The interbody device 410 may include one or more (e.g., two) tool interfaces 414. The tool interfaces may be configured to be grasped by, attach to, or otherwise be contacted and engaged by a tool (not shown) during a medical implant procedure. [00119] The interbody device 410 may be formed as a single piece (not shown), or it may be assembled from two or more pieces, such as, for example the frame 413 and a pair of the locking elements 247.

The body or frame 413 of the interbody device 410 may include antimigration and/or anchoring features 4132. The features 4132 may be configured to contact and engage surface portions of, for example, the cage 102 (shown in FIGS. 17B-17E) to secure the interbody device 410 to the cage. The features 4132 may be configured to assist in aligning and proper positioning of the interbody device 410 with respect to the cage 102 in a manner similar to that described above with respect to the interbody device 240 and cage 102 (shown in FIG. 15E). The features 4132 may be configured similar to or substantially the same as the features 2432 described above.

The frame 413 may include anti-migration and/or anchoring features (not shown) located on upper and/or lower surfaces of the interbody device 410 to contact and engage adjacent bone surface(s). The features may comprise, for example, a pattern and/or texture that provides anti-migration and/or anchoring characteristics when implanted in the spine 2. The features may comprise, e.g., teeth, serrations, protrusions (e.g., triangular, pyramidal, conical, semi spherical, quadrilateral, rectangular, cylindrical, diamond, elliptical, and/or irregular shapes, or the like), or the like.

The frame 413 may include a channel (not shown) similar to the channel 2433 shown in FIGS. 13A and 13C. The channel may be provided on one or both inner walls of the frame 413. The channel may be configured to receive, engage and guide the plate guide 146 of cage 102 (shown in FIG. 4) in a manner similar to or substantially the same as the channel 2432 described above, thereby providing proper alignment and positioning of the interbody device 410 with respect to the cage (e.g., shown in FIG. 17B).

As noted previously, the locking element 247 may be similar in construction and manner of use as described, for example, in FIGS. 3A-22D, 33, 35, 37, 39, 55, 58-65B, or 69A-78E and the corresponding text in U.S. patent application Ser. No. 14/956,084, filed Dec. 1, 2015, titled “Intervertebral Implants and Related Systems and Methods,” which has been incorporated herein by reference. Further, various arrangements of interbody device 410 may include one or more features configured to facilitate sagittal and/or coronal visibility. For example, the body or frame 413 of interbody device 410 may comprise a radiopaque material visible via x-ray or similar forms of imaging modalities. As such, frame 413 may enable accurate positioning and/or placement of interbody device 410 within and/or along spinal column 2 (shown in FIG. 1).

The interbody device 410 may further include an engager element 2431 on at least one side of the frame 413, which may function in the manner described above with references to FIGS. 13A and 13B. The engager element 4131 may function in the same manner as the engager element 2431.

The interbody device 410 may be implanted in a manner similar to that described above for interbody device 240, with references to FIGS. 13A-13C and 14. The interbody device 410 may be configured for use in, for example, anterior approach and discectomy applications FIGS. 17B-17E illustrate various views of an example of an interbody system 400 that includes the interbody device 410. As seen, the interbody system 400 includes the interbody device 410 and the cage 102 (shown in FIG. 4). The interbody system 400 may be formed as a single structure (not shown), or it may be assembled from two or more pieces.

For instance, referring to FIG. 17B, the interbody system 400 may be assembled by attaching the interbody device 410 to the cage 102 (or cage 101, shown in FIG. 3). In this regard, the interbody device 410 may be assembled in a manner similar to or substantially the same as that described above with references to FIG. 15E for the interbody system 300.

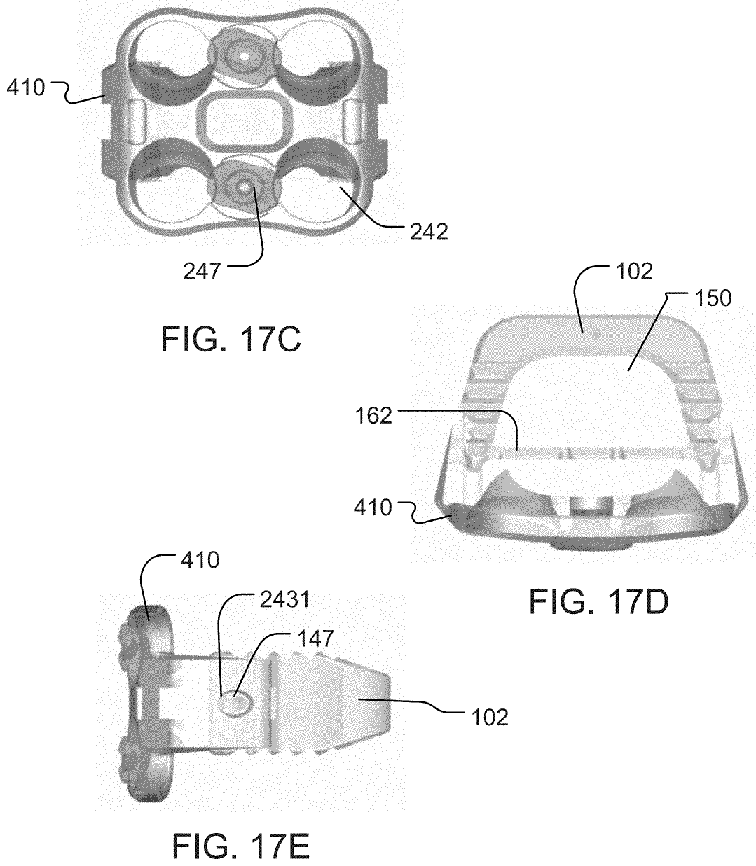

FIG. 17C shows an example of the interbody system 400 with the locking elements 247 positioned in a locking or near-locking position. As seen, a portion of the locking elements 247 is turned and positioned in the aperture(s) 242, thereby partially (or entirely) blocking the aperture(s) 242, so that the bone fastener 11 (shown in FIG. 18B) is prevented from backing out of the aperture 242.

FIG. 17D shows a superior (or inferior) view of the interbody system 400.

FIG. 17E shows a sagittal view of the interbody system 400.

FIGS. 18A-18C illustrate an example of implanting the interbody system 400 between a pair of bony structures (e.g., vertebrae) 4.

Referring to FIGS. 17B-17E and 18A-18C simultaneously, the interbody system 400 may be configured for use in, for example, anterior approach and discectomy applications. For instance, after a patient is positioned in a supine position on, for example, a radiolucent operating table, the surgical area cleaned, an incision made, muscle tissue and/or organs moved to the side(s), and other common surgical procedures carried out, a disc may be incised, removed, and the space prepared for implanting of an interbody system 400. The bone surfaces and edges on the adjacent vertebrae 4 may be carefully contoured, as appropriate.

When desired, a medical professional may determine an appropriate size of the interbody device 410 and/or the cage 102 (or 101) by selecting an appropriately dimensioned interbody device 410 and/or an appropriately dimensioned cage 102 (or 101), each of which may be selectable based on, for example, height, width, depth, surface angle(s), and the like. Where the interbody system 400 is provided as a single unit, the interbody system 400 as a unit may be selected based on its dimensions for the particular application.

Upon selecting the appropriate interbody system 400 (e.g. interbody device 410 and cage 102), one or more of an ACIF, ALIF, or the like may be performed by placing the interbody system 400 between adjacent vertebrae 4 in the space formed by the removed degenerated disc (shown in FIGS. 18A-18C). The medical professional may then place the cage portion of the interbody system 400 in the space between the vertebrae 4. The medical professional may prepare the coronal surfaces of the adjacent vertebrae 4 by removing bone material to substantially match the outer perimeter of the interbody device 410, so as to receive at least a portion of the interbody device 410 in the prepared areas on the vertebrae 4 and thereby position the anterior coronal face 401 of the interbody device 410 (shown in FIG. 17A) substantially flush with the anterior coronal surfaces of the adjacent vertebrae 4.

Once the interbody system 400 is seated in its final position, four bone fasteners 11 may be installed using an instrument (not shown), such as, for example, a screwdriver (not shown). As each fastener 11 is inserted through the aperture 242 and into contact with the wall membrane 162, the wall membrane 162 may bend and provide directional support against the skyping due to the springboard effect of wanting to back into its natural state. Simultaneously, due to the pushing of the wall membrane 162 into the graft chamber 150, the wall membrane may direct graft material from the graft chamber 150 to the areas surrounding the interbody system 400.

After the fasteners 11 are implanted in their final positions in the anchoring sites, the locking element 247 may then be turned or otherwise manipulated to secure the fasteners 11 in place, thereby preventing the fasteners 11 from loosening or withdrawing from their respective anchoring sites.

After the bone graft materials are installed, and the bone fasteners 11 may be securely and properly placed, and the installation of the interbody system 400 (or 300) completed, the area may be cleaned, checked, closed and other post-operative procedures carried out, as is known in the art.

Placement of the interbody system 400 within spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, such interbody system 400 may facilitate fusion (e.g., bone to grow together) between adjacent vertebrae 4 by stabilizing adjacent vertebrae 4 relative to one another.

In the instant disclosure, where the fastener 11 includes a bone screw, a thread may be tapped into the bone to form a tap (not shown) to receive and securely hold the bone fastener 11. The process would be repeated for each fastener 11. Such holes may be formed with the aid of a separate drill guide (not shown) positioned proximate or abutting vertebra 4 and inserting a drill therethrough. Alternatively, such holes may be formed free hand, without the use of a drill guide.

In various embodiments, after the interbody device or interbody system is properly installed with respect to the vertebrae 4 (e.g., as shown in FIGS. 14, 16A-16C or 18A-18C), the bone fastener(s) 11 may be installed. In this regard, a driver tool (not shown), as is known by those skilled in the art, may be used to turn and drive the bone fastener(s) 11 into the vertebrae 4. It is noted that the bone fastener(s) 11 may be aligned with the tap (not shown) in the bone and screwed into the threaded tap. Alternatively, the bone fasteners 11 may be partially installed in the tap before being contacted by the driver tool. Once the bone fasteners 11 are implanted in the desired position, the driver tool may be removed and the process repeated for each bone fastener 11.

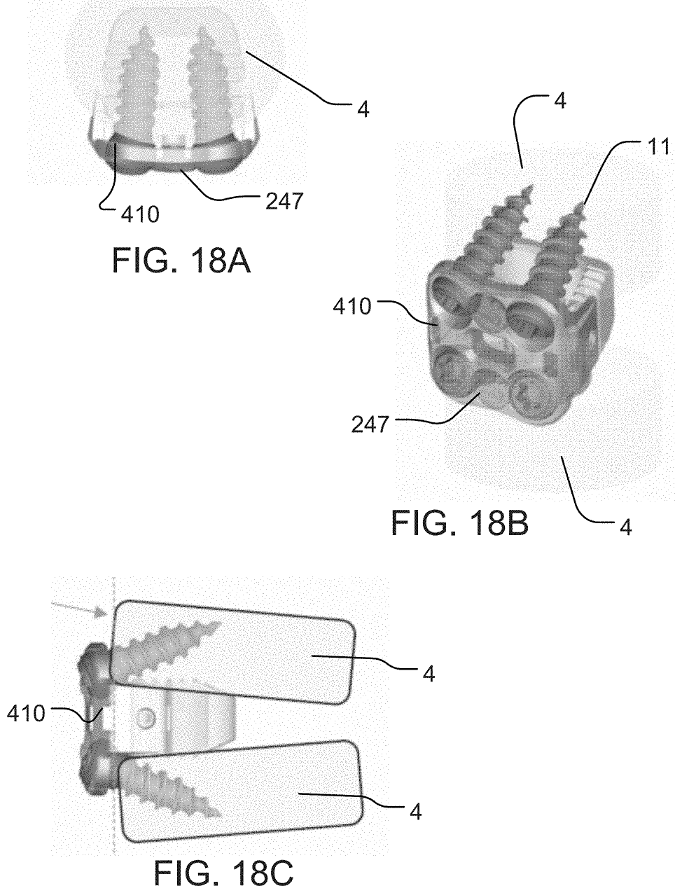

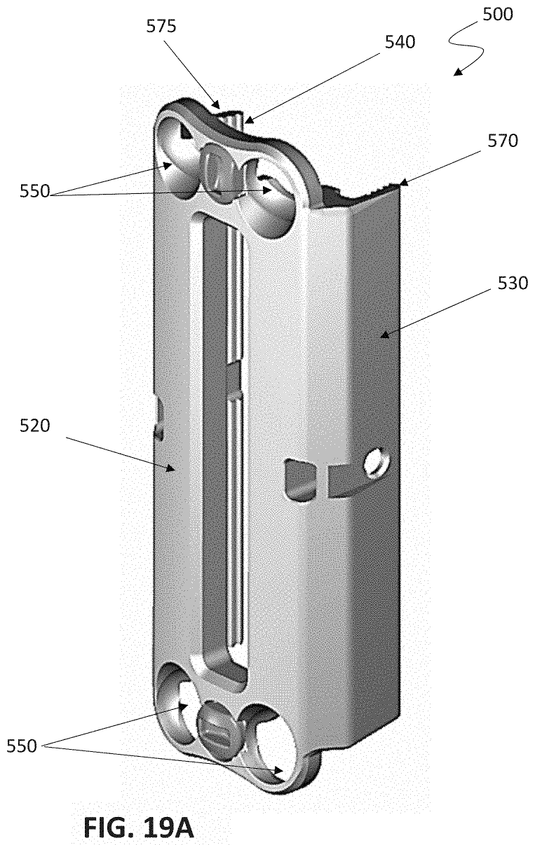

FIGS. 19A through 19F depict another exemplary embodiment of an implant device for use in various spinal surgical procedures. In this embodiment, a corpectomy plate 500 is disclosed, which should be understood to be just one exemplary embodiment—other different embodiments are contemplated and are within the scope of this application. The plate 500 can include a central body 520 having a roughly planar or plate-like shape, with a first “fin” or first extension arm 530 and a second “fin” or second extension arm 540 extending outward from lateral edges of the central body. Desirably, the plate 500 can include one or more openings 550 to accommodate fixation devices such as screws 555 (see FIG. 19G), which can be utilized to attach and secure the plate 500 to adjacent bony anatomy. In the disclosed embodiment the plate 500 can include four openings 550, with an upper pair of openings 550 to accommodate attachment to a superior vertebrae and a lower pair or openings 550 to accommodate attachment to an inferior vertebrae.

As best seen in FIG. 19D, the central body 520 can include a bone-facing inner surface 560, with the first and second extension arms 530 and 540 desirably extending from the central body towards the bone-facing direction. In various embodiments, the first extension arm 530 can include an upper surface 570 for engaging with a superior vertebrae, and the second extension arm 540 can have an upper surface 575 for engaging with the superior vertebrae. Similarly, the first and second extension arms 530 and 540 can have respective lower surfaces 580 and 585 for engaging with an inferior vertebrae.

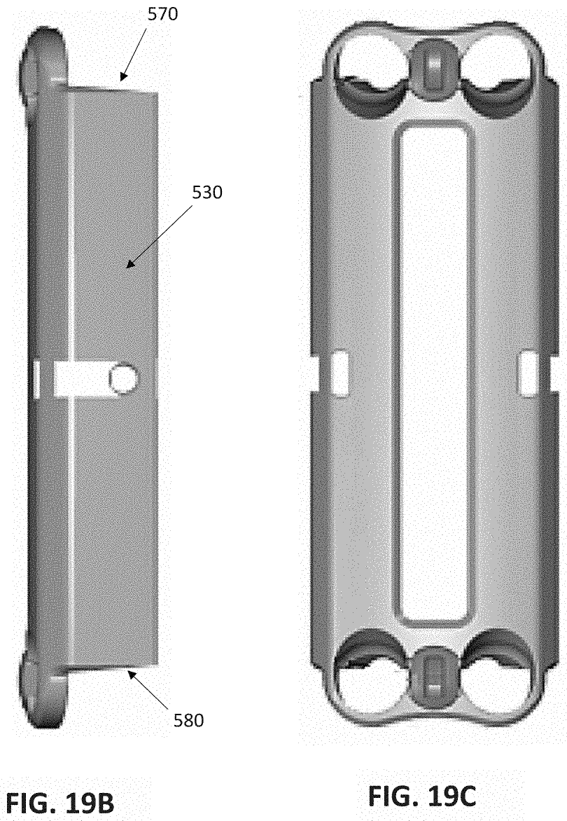

FIG. 20 depicts one exemplary embodiment of a corpectomy plate 600 positioned in a simplified representation of a vertebral column. In this embodiment, a central vertebrae 610 (indicated in dotted lines) has been removed from between a superior vertebrae 605 and an inferior vertebrae 615, and a bone block 620 or similar graft material has been positioned between the superior and inferior vertebrae 605 and 615. The corpectomy plate 600 can be secured adjacent to the bone block 620, with first and second extension arms extending into the space between the superior and inferior vertebrae 605 and 615, and in various embodiments can encompass some portion of the bone block 620 therebetween. Desirably, upper and/or lower surfaces on the first and second extension arms can engage with bone surfaces of the superior and inferior vertebrae 605 and 615, and fixation screws 640 can be secured to the vertebral bodies in a known manner. If desired, the corpectomy plate 600 can be secured directly to the bone block with screw or other fixation (not shown), or the first and second extension arms can include inwardly extending engagement features 590 that attach to the bone block, if necessary (see FIG. 19F).

The engagement and/or attachment between the plate and the bone block or intervertebral spacer can take a variety of forms, depending upon the construction and design of the various system components. In some embodiments, the bone block or other implant can be captured between the extension arms by compression and/or flexion of the arms to some desired degree, while in other embodiments fixation between the plate and any appropriate intervertebral spacer or graft material can be accomplished using a snap fit arrangement or complimentary shaped forms (i.e., dovetail and/or jigsaw-piece configurations). In other embodiments, screw or other fixation could connect the plate to the intervertebral spacer or graft material.

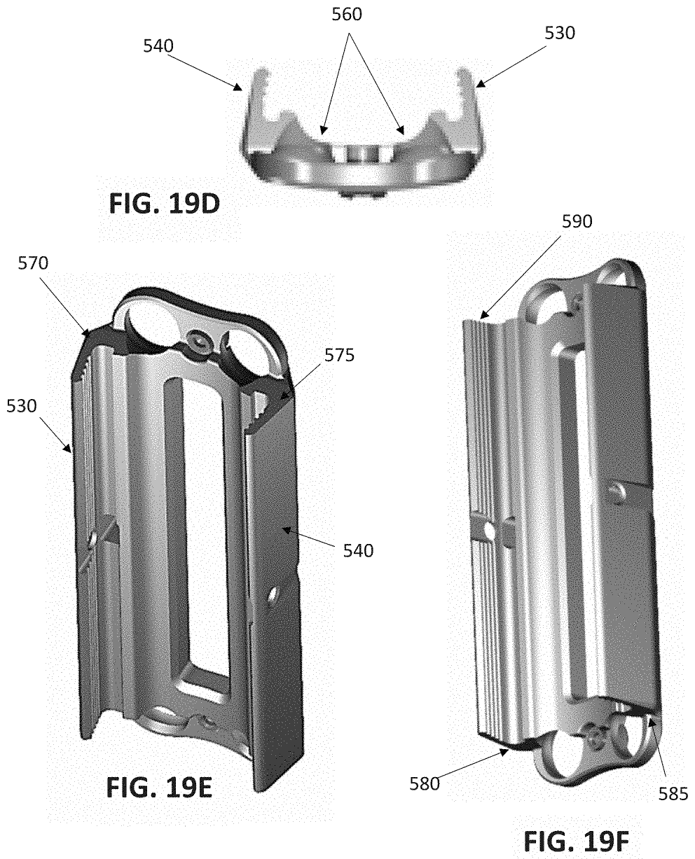

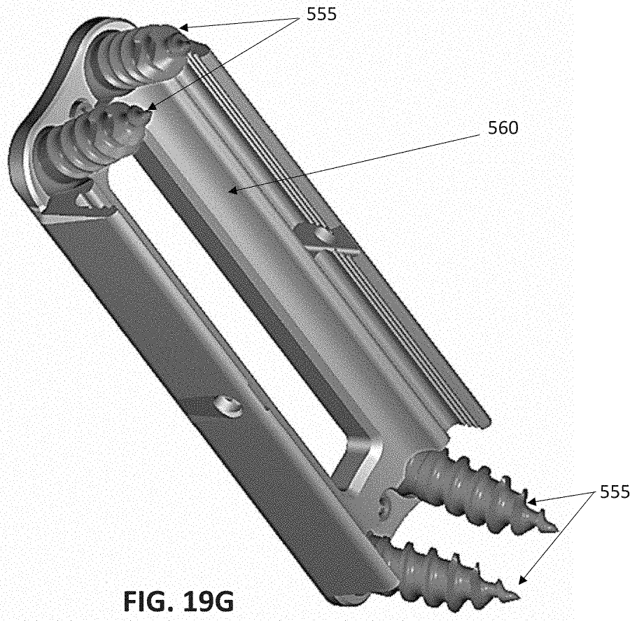

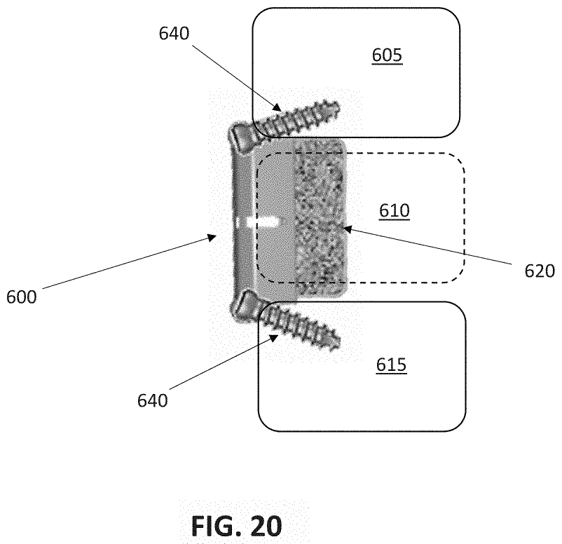

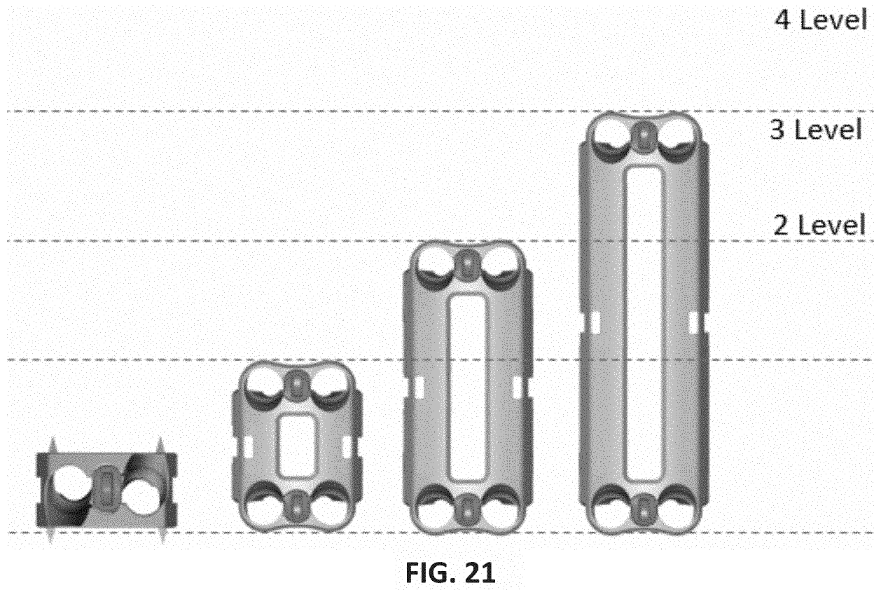

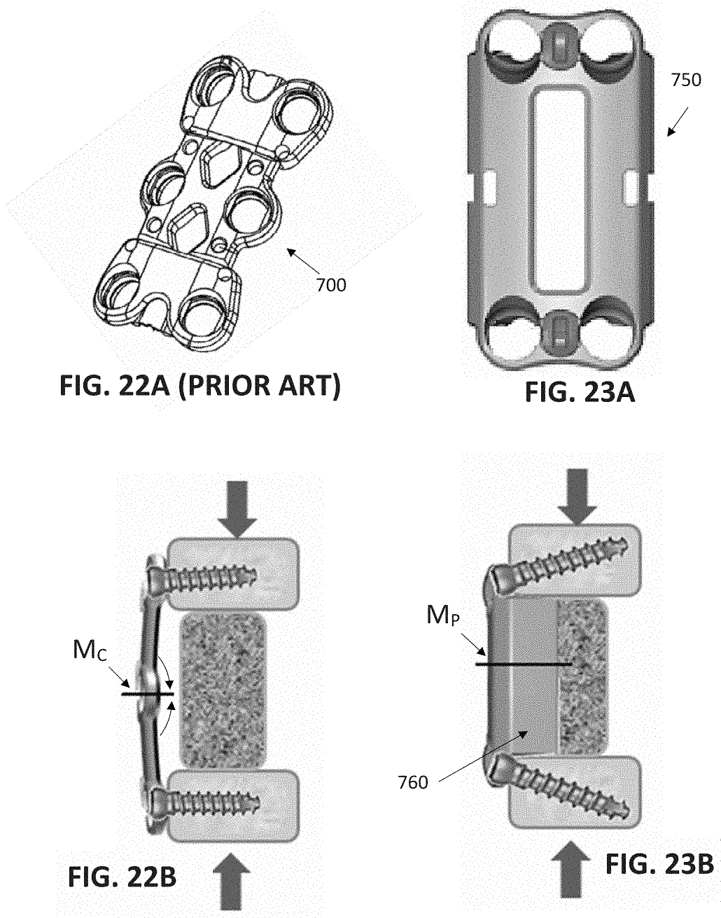

FIG. 21 depicts a variety of embodiments of corpectomy plates, with some of the embodiments sized and/or particularly well suited for replacement and/or support of multiple levels of vertebral bodies, including where multi-level corpectomies have been performed. For example, such implants and/or plating devices could range from 6 mm in height up to 70 mm in height (or greater) to accommodate one, two, three and/or four level vertebral replacement and/or augmentation procedures. In some embodiments, placement and/or number of openings and/or other features in the more central regions of the plates may be minimized to optimize plate rigidity. Desirably, the C-Beam cross-sectional geometry of the corpectomy plate will provide a pair of reinforcing “ridges” that provide supplemental support the plate while also increasing surface contact area with the upper and lower vertebral endplates. As best seen in FIGS. 22A and 22B, the compressive loading of a prior art 6-hole plate construct 700 in a corpectomy application tends to create a significant moment force centered on a midpoint Mc of the construct 700, and it is in this location where such constructs typically fracture and fail. In contrast, as shown in FIGS. 23A and 23B, the extension arms 760 of the present disclosed corpectomy plate 750 are designed to distribute such compressive loading over a much larger surface area of the plate, which significantly reduces any resulting stress concentrations at the midpoint M_{P} of the plate 750, thereby desirably preventing failure of the plate 750 at this location.

It should be understood that the various embodiments of a corpectomy cage described herein may be utilized as a stand-alone cage for some surgical applications, and may be utilized in conjunction with various other components (i.e., interbody cages, bone graft blocks and/or vertebral body replacement devices) in other surgical applications. In a similar manner, the various other components (i.e., interbody cages, bone graft blocks and/or vertebral body replacement devices) may be utilized a stand-alone treatments in some surgical applications, and in conjunction with various combinations of the other components described herein (including, but not limited to, the various corpectomy cage designs disclosed herein) in other surgical applications.

In various alternative embodiments, one or more bone-facing surfaces of the corpectomy plates described herein could include a textured engagement surface that bears against the respective vertebra, if desired. The engagement surface may be textured in any suitable manner, including teeth-like projections or other texturing. For example, the texturing may mimic the texturing of natural bone surface, or may comprise a bone-ingrowth surface. Such surface features could be accomplished via 3-D material building (e.g., 3-D printing), including the employment of ceramics, plastics and/or metals, such as titanium and stainless steel, or other any other material for such 3-D material building.

Various locking/securing mechanisms/means, if desired, are contemplated to help retain the device in a specific adjustment (e.g., at least some distance of the respective engagement areas are adjusted).

In various embodiment, method(s) for manufacturing the disclosed plating devices and/or implanting the device into a spine are contemplated and are part of the scope of the present application.

The terms “including,” “comprising,” and variations thereof, as used in this disclosure, mean “including, but not limited to,” unless expressly specified otherwise. The terms “a,” “an,” and “the,” as used in this disclosure, means “one or more,” unless expressly specified otherwise.

Devices that are in communication with each other need not be in continuous communication with each other, unless expressly specified otherwise. In addition, devices that are in communication with each other may communicate directly or indirectly through one or more intermediaries.

Although process steps, method steps, or the like, may be described in a sequential order, such processes and methods may be configured to work in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure.

1. An implant device for the spine, the implant device for location at least partially between two adjacent vertebrae, comprising:
an elongated spinal plate assembly having an upper surface, a lower surface, a front surface and a back surface, at least a portion of the back surface comprising a bone-facing surface, the elongated plate having a first opening proximate to the upper surface of the plate, the first opening extending from the front surface to the back surface of the plate, the elongated plate further having a second opening proximate to the lower surface of the plate, the first opening extending from the front surface to the back surface of the plate, the elongated plate having a first elongated arm extending outward from the back surface in a first direction away from the front surface and a second elongated arm extending outward from the back surface in a second direction away from the front surface, the first and second elongated arms spaced apart from each other, the first and second elongated arms each having a first upper bone facing surface and a second lower bone facing surface.

2. The implant device of claim 1, wherein the elongated spinal plate comprises titanium.

3. The implant device of claim 1, wherein the elongated spinal plate assembly further includes a centrally located opening which extends from the front surface to the back surface of the plate, and the first and second elongated arms are positioned laterally from the centrally located opening.

4. The implant device of claim 3, wherein the centrally located opening extends along a longitudinal axis of the elongated spinal plate assembly.

5. The implant device of claim 3, wherein a longitudinal length of the centrally located opening is less than a longitudinal length of at least one of the first and second elongated arms.

6. The implant device of claim 1, wherein the first elongated arm includes a first textured surface and the second elongated arm includes a second textured surface.

7. The implant device of claim 6, wherein the first and second textured surfaces face towards each other.

8. The implant device of claim 6, wherein the first upper bone facing surfaces of the first and second elongated arms are substantially parallel.

9. The implant device of claim 1, wherein the at least a portion of the back surface comprises a first bone facing back surface located proximate to the upper surface of the plate, the first bone facing surface being substantially perpendicular to the first upper bone facing surface of the first elongated arm.

10. The implant device of claim 1, further comprising:
an intervertebral cage structure comprising:
a main body comprising a first surface and a second surface located opposite to the first surface;
an opening formed in the intervertebral cage structure and extending from the first surface to the second surface located opposite the first surface of the main body,
wherein at least a portion of the intervertebral cage structure is sized and configured to fit at least partially between the first and second elongated arms.

11. The implant device of claim 10, wherein the first elongated arm includes a first textured surface and the second elongated arm includes a second textured surface, and the main body includes an exterior surface that engages with and is retained by the first and second textured surfaces.

12. The implant device of claim 1, wherein at least a portion of the bone facing surface comprises silicon nitride.

13. The implant device of claim 1, wherein at least a portion of the first and second elongated arms comprises silicon nitride.

14. A spinal implant comprising:
a spacer configured for retention between a first vertebrae and a second vertebrae, the spacer having a cavity; and
a spine plate coupled to the spacer, the spine plate comprising:
a first spine plate portion having a first end and a second end, the first end having a first bone screw bore to allow attachment of the first spine plate portion to the first vertebrae via a first bone screw, and the second end having a second bone screw bore to allow attachment of the first spine plate portion to the second vertebrae via a second bone screw,
a first fin extending outward from the first spine plate portion, the first fin positioned laterally of the first and second bone screw bores and extending substantially parallel to an axis of the first bone screw bore, the first fin including a first engagement surface for engaging an external surface of the spacer.

15. The spinal implant of claim 14, further comprising a second fin extending outward from the first spine plate portion, the first and second bone screw bores positioned between the first and second fins, the second fin including a second engagement surface for engaging an external surface of the spacer.

16. The spinal implant of claim 15, wherein a distal end of the first fin and a distal end of the second fin are canted towards each other.

17. The spinal implant of claim 14, wherein the spine plate comprises titanium and the spacer comprises a natural bone material.

18. The spinal implant of claim 14, wherein the spine plate comprises titanium and the spacer comprises silicon nitride.

19. The spinal implant of claim 15, wherein the first spine plate portion further includes an elongated opening positioned between the first and second fins.

20. The spinal implant of claim 14, wherein the spine plate and spacer are coupled together prior to insertion of the spinal implant into the patient's anatomy.
